(19) United States
(12) Patent Application Publication
(10) Pub. No.: US 2020/0375849 A1
(43) Pub. Date: Dec. 3, 2020

(54) DEVICES AND METHODS FOR PROVIDING MEDICATION

(71) Applicant: CMPD Licensing, LLC, Conroe, TX (US)

(72) Inventor: Jay Richard Ray, II, Conroe, TX (US)

(73) Assignee: CMPD Licensing, LLC, Conroe, TX (US)

(21) Appl. No.: 16/423,937

(22) Filed: May 28, 2019

(51) Int. Cl. A61J 7/00; A61J 1/20; A61K 31/5383; A61K 31/496; A61J 1/10; A61K 9/00

(52) U.S. Cl. CPC A61J 7/0053; A61J 1/20; A61K 31/5383; A61J 2205/40; A61J 1/10; A61K 9/0053; A61K 31/496

(57) ABSTRACT

Supplying a medication for use with a medication delivery system includes supplying a liquid medication composition into an interior volume of a drug chamber and dispensing the drug chamber to a patient or caregiver. The drug chamber may include a fitting for coupling to a corresponding fitting of a transfer module and a drug port. The transfer module may also include a transfer port configured to align with the drug port when the drug chamber and transfer module are coupled at their fittings. The transfer module may be operable to open the drug port to allow transfer of a portion of the liquid medication composition through the transfer port into a separate dose chamber.

### TECHNICAL FIELD

The present description is directed to devices and methods for providing medication, more specifically, the present description is directed to transfer of medication for preparation of an administration dosage.

### BACKGROUND

Prescription medication is typically dispensed by a pharmacy. At the pharmacy, patients may receive instructions regarding preparation and administration. For example, patients may be instructed to prepare a dosage amount such as a specified number of pills or a volume of solution to ingest or apply.

It is often difficult or inconvenient for patients to visit a pharmacy to obtain prescription medication. Mail order dispensing has gained popularity. However, with mail order dispensing of medication, patients may not receive sufficient guidance with respect to preparation and administration of the medication. Measuring dosages can also be complex and difficult even with proper instruction for patients that lack the proper measurement devices, coordination, or mobility. Whether mail order or obtained at a pharmacy, stability of medications and protecting children for accessing the medication are also concerns. The above concerns also apply to over-the-counter medications. What is need are improved methods for preparing medication dosages.

### SUMMARY

In one aspect, a method of supplying a medication for use with a medication transfer system includes supplying a liquid medication into an interior volume of a drug chamber and dispensing the drug chamber to a patient or caregiver. The drug chamber may include a fitting for coupling to a corresponding fitting of a transfer module. The drug chamber may also include a drug port. The transfer module may also include a transfer port configured to align with the drug port when the drug chamber and transfer module are coupled at their fittings. The transfer module may be operable to open the drug port to allow transfer a portion of the liquid medication through the transfer port into a separate dose chamber.

The liquid medication may comprise a prescription medication selected from an anti-infective and/or corticosteroid. In one example, the liquid medication comprises levofloxacin or itraconazole. In a further example, the liquid medication comprises levofloxacin 25 mg/mL oral solution. In one example, the liquid medication comprises itraconazole 10 mg/mL oral solution.

The drug chamber may include a hose that extends from within its interior volume to the drug port.

In another aspect, a method of preparing a measured medication dosage includes transferring a transfer volume of liquid medication from a drug chamber through a transfer port and into a dose chamber. A transfer module may be positioned to at least partially control transfer of the liquid medication through the transfer port. The transfer volume may be less than the volume of liquid medication within the drug chamber when transfer begins.

The dose chamber may be configured for measurement of the transfer volume. In one example, the dose chamber includes measurement markings corresponding to the transfer volume.

The transfer module may be configured for measurement of the volume of transferred medication. In one example, the transfer module includes a user interface including a control actuator and transferring the liquid medication includes actuating the control actuator to initiate the transfer of the liquid medication. The drug chamber may be child-resistant.

The liquid medication is a prescription medication comprising an anti-infective and/or corticosteroid. In one example, the liquid medication comprises levofloxacin 25 mg/mL oral solution, itraconazole 10 mg/mL oral solution, or both.

In still another aspect, a method includes supplying a medication together with a medication transfer system including dispensing a medication transfer device. The medication transfer device includes a drug chamber having an interior volume, a dose chamber having an interior volume, and a transfer module including a transfer port between the interior volumes of the drug chamber and the dose chamber. The method may further include dispensing liquid medication within the interior volume of the drug chamber when dispensing the drug chamber. The liquid medication may be selectively transferable from the interior volume of the drug chamber to the interior volume of the dose chamber. The medication transfer device may be configured for measurement of a volume of the transferred liquid medication.

In one configuration, the medication transfer device comprises a bag that is partitioned to define the drug chamber and dose chamber. The medication transfer device may comprises a child-resistant device. In one example, the medication transfer device comprises a child-resistant bottle or child-resistant bag.

In one example method, the liquid medication is a prescription medication comprising an anti-infective and/or corticosteroid. The liquid medication may be levofloxacin 25 mg/mL oral solution, itraconazole 10 mg/mL oral solution, or both.

### BRIEF DESCRIPTION OF THE DRAWINGS

The novel features of the described embodiments are set forth with particularity in the appended claims. The described embodiments, however, both as to organization and manner of operation, may be best understood by reference to the following description, taken in conjunction with the accompanying drawings in which:

FIG. 1 semi-schematically illustrates a medication transfer device of a medication delivery system according to various embodiments described herein;

FIG. 2 semi-schematically illustrates a medication transfer device of a medication delivery system including measurement markings according to various embodiments described herein;

FIG. 3 semi-schematically illustrates a medication transfer device of a medication delivery system according to various embodiments described herein;

FIG. 4 semi-schematically illustrates a medication transfer device of a medication delivery system including multiple transfer modules according to various embodiments described herein;

FIG. 5 semi-schematically illustrates a medication transfer device of a medication delivery system including multiple dose chambers and transfer modules according to various embodiments described herein;

FIG. 6 semi-schematically illustrates a medication transfer device of a medication delivery system including multiple drug chambers and transfer modules according to various embodiments described herein;

FIG. 7 semi-schematically illustrates a medication transfer device of a medication delivery system including a dose release port according to various embodiments described herein;

FIG. 8 semi-schematically illustrates a medication transfer device of a medication delivery system including a dose release port according to various embodiments described herein;

FIG. 9 semi-schematically illustrates a medication transfer device of a medication delivery system including a dose release port according to various embodiments described herein;

FIG. 10 semi-schematically illustrates a medication transfer device of a medication delivery system including a fill port according to various embodiments described herein;

FIG. 11 semi-schematically illustrates a medication transfer device of a medication delivery system including a fill port according to various embodiments described herein;

FIG. 12 semi-schematically illustrates a medication transfer device of a medication delivery system including a fill port according to various embodiments described herein;

FIG. 13 semi-schematically illustrates features of a transfer module according to various embodiments described herein;

FIG. 14 semi-schematically illustrates features of a transfer module according to various embodiments described herein;

FIG. 15 semi-schematically illustrates a compressible medication transfer device of a medication delivery system according to various embodiments described herein;

FIG. 16 semi-schematically illustrates a medication transfer device of a medication delivery system including a movable partition according to various embodiments described herein;

FIG. 17 semi-schematically illustrates a medication transfer device of a medication delivery system including a pressure actuator according to various embodiments described herein;

FIG. 18 semi-schematically illustrates a medication transfer device of a medication delivery system including a pressure actuator according to various embodiments described herein;

FIGS. 19A & 19B semi-schematically illustrate features of a transfer module according to various embodiments described herein;

FIGS. 20A-20C semi-schematically illustrate features of a transfer module according to various embodiments described herein;

FIG. 21A-21C semi-schematically illustrate features of a transfer module according to various embodiments described herein;

FIG. 22 illustrates a medication transfer device of a medication delivery system according to various embodiments described herein;

FIG. 23 illustrates a compressible medication transfer device of a medication delivery system according to various embodiments described herein;

FIG. 24 illustrates a medication transfer device of a medication delivery system according to various embodiments described herein;

FIG. 25 illustrates a medication transfer device of a medication delivery system including measurement markings according to various embodiments described herein;

FIG. 26 illustrates a medication transfer device of a medication delivery system including a lid according to various embodiments described herein;

FIG. 27 illustrates a modular medication transfer device of a medication delivery system according to various embodiments described herein;

FIG. 28 illustrates a modular medication transfer device of a medication delivery system according to various embodiments described herein;

FIG. 29 illustrates a modular medication transfer device of a medication delivery system according to various embodiments described herein;

FIG. 30 illustrates a medication transfer device of a medication delivery system according to various embodiments described herein;

FIG. 31 illustrates a modular medication transfer device of a medication delivery system according to various embodiments described herein;

FIG. 32 illustrates a modular medication transfer device of a medication delivery system according to various embodiments described herein;

FIG. 33 illustrates a modular medication transfer device of a medication delivery system according to various embodiments described herein; and

FIG. 34 illustrates a modular medication transfer device of a medication delivery system according to various embodiments described herein.

### DESCRIPTION

The medication delivery system may comprise a medication transfer device including a drug chamber for containing medication and a dose chamber for receiving a dose, which may be a full or partial dose, of the medication. The medication transfer device may also include a transfer module for transferring medication from the drug chamber to the dose chamber. The transfer module may be operable to transfer or enable transfer of a transfer volume of medication. One or more transfer volumes will typically provide a dosage volume suitable for administration to a patient. The medication transfer device may be configured to contain multiple dosages of medication within the drug chamber and be operable to transfer a predetermined and/or customized transfer volume of the medication into the interior volume of the dose chamber. Various transfer mechanisms, metering mechanisms, or combinations thereof may be used. In some embodiments, one or more transfer volumes or dosage volumes correspond to the interior volume of the dose chamber. The dose chamber may also include measurement markings from which desired transfer volumes may be determined.

The medication transfer device may include various configurations such as a bag, bottle, jar, vial, or pouch. The medication transfer device or chambers thereof may be formed from generally liquid impermeable material such as glass; plastic such as PVC, vinyl, polypropylenes (PP), polyethylenes (PE), polyethylene terephthalates (PET) such as boPET, or other plastics; ceramic; metal; or combinations thereof.

The medication transfer device may also include various child-resistant features. For example, access to the drug chamber, dose chamber, and/or operations of the transfer module may be equipped with child-resistant features that are resistant to child usage, e.g., in compliance with the Poison Prevention Packaging Act or as otherwise with the Federal Drug Administration or U.S. Consumer Product Safety Commission. Examples may include caps, lids, or over caps utilizing local squeeze force applied while turning for removal or operation, continuous threads, hold fitment while turning, push down while turning, push down into limited fitment gap followed by turn to secondary fitment gap for access to removal or operation functions, lug finish closures, align cap and ring followed by pushing cap toward ring and turning, localized push up to remove, localized press down then pull up at arrow, turn cap to stop and then lift and continue opening, localized push in while turning where force must be applied to designated place on closure, or other child-resistance mechanisms, which may include others described herein. Additional child-resistant features may include trigger pumps (press down on a point to release lock and rotate orifice to spray position to squeeze trigger), finger pumps (push tab while rotating to spray position, zipper or seal locks).

In various embodiments, a method of providing a medication comprises providing the medication delivery system, medication transfer device, or a component thereof. In one example, the method comprises providing a drug chamber, containing medication and configured for coupling with the transfer module and dose chamber. In another example, the method includes providing a refill container for charging the drug chamber with medication. In one example, the method includes providing a transfer module operable to provide a desired transfer volume when coupled between the drug chamber and the dose chamber. In some embodiments, the method includes providing a drug chamber containing medication and an attached or attachable transfer module for coupling with a dose chamber. The transfer module may be configured to provide a suitable transfer volume or may be configured to allow a user to select a transfer volume, which may comprise multiple transfer volumes. In one embodiment, multiple interchangeable transfer modules may be provided, each configured for use in transferring a different transfer volume.

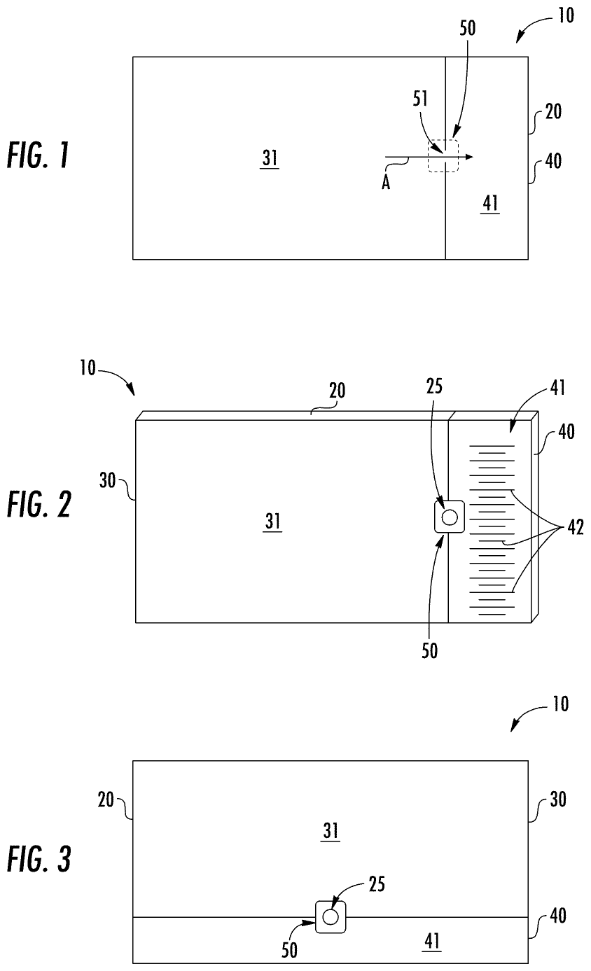

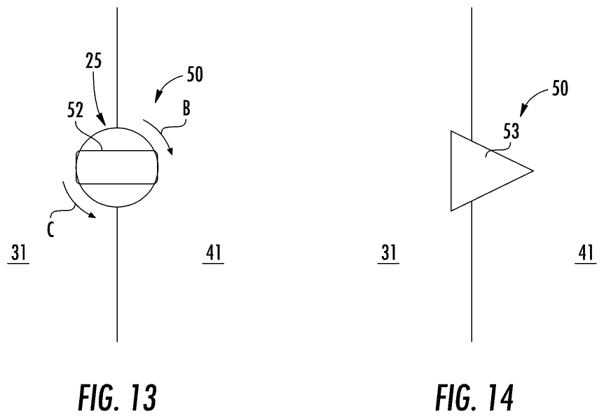

FIGS. 1-34 illustrate various embodiments of a medication delivery system 10 wherein similar features are identified with similar numbers. The medication delivery system 10 may include a medication transfer device 20 comprising a drug chamber 30 and a dose chamber 40, each dimensioned to define a respective interior volume 31, 41. The medication transfer device 20 may comprise a transfer module 50 for providing transfer of medication. The transfer module 50 may be utilized to control transfer of medication through a transfer port 51 and may include various transfer valves and/or metering structures (see, e.g., FIGS. 13-16 & FIGS. 21A-21C) for controlling transfer of medication. The medication transfer device 20 or chambers 30, 40 thereof may be formed from generally liquid impermeable material such as plastic, metallized plastic, or other suitable bag material, such as those identified elsewhere herein. The drug chamber 30 may be configured to be in fluid communication or selectively in fluid communication with the dose chamber 40 via a transfer port 51 for transferring medication contained within the drug chamber 30 to the dose chamber 40, e.g., as indicated by arrow A in FIG. 1.

The medication transfer device 20 may be configured to contain multiple dosages of medication within the drug chamber 30 and is operable to transfer a volume of the medication into the interior volume 41 of the dose chamber 40. The interior volume 41 of the dose chamber 40 may define a desired dosage such that transferring an amount of medication, e.g., one or more transfer volumes, to fill the dose chamber 40 provides the desired measured dosage volume. In some such embodiments, a medication delivery system 10 may comprise a plurality of medication transfer devices 20 having different sized dose chambers 40 for providing different dosage volumes. In some embodiments, as described in more detail below, a medication transfer device 20 may include multiple dose chambers 40, each in fluid communication or selectively in fluid communication with one or more drug chambers 30. For example, multiple dose chambers 40 having the same or different interior volumes 41 may be used. Multiple drug chambers 30 may be provided to supply the same or different medications to one or more dose chambers 40. For example, a medication transfer device 20 may include a first drug chamber 30 for holding a first medication transferable to a dose chamber 40 or intermediate chamber and a second drug chamber 30 for holding a second medication transferable to the dose chamber 40, a different dose chamber 40, or the intermediate chamber. The intermediate chamber may be used for mixing before transfer to a dose chamber 40, which may include mixing additional medication before or after transfer.

It is contemplated that any of the various features described with respect to drawings may be utilized in combination with other described features describes with respect to other embodiments. However, various features are described separately for ease of understanding. Furthermore, while certain features are described with respect to a bag, bottle, or container configuration, those having skill in the art will appreciate that these features may find use in other device configurations, such as those described herein, and their use in such other device configurations is contemplated herein.

FIGS. 1-21C illustrate various features of a medication delivery system 10 including a medication transfer device 20 comprising a bag configuration.

With particular reference to FIG. 1, the medication transfer device 20 comprises a partitioned bag including a transfer module 50 for providing transfer of medication through a transfer port 51 from the interior volume 41 of the drug chamber 30 to the interior volume 41 of the dose chamber 40. The chambers 30, 40 may comprise generally liquid impermeable material such as glass; plastic such as PVC, vinyl, polypropylenes (PP), polyethylenes (PE), polyethylene terephthalates (PET) such as boPET, or other plastics; ceramic; metal; or combinations thereof.

The medication transfer device 20 is configured to contain multiple dosages of medication within the drug chamber 30 and is operable to transfer a transfer volume of the medication into the interior volume 41 of the dose chamber 40. The transfer volume may be a predetermined, selectable, or customizable volume of medication according to a configuration of the transfer module 50 or device configuration, as described in more detail elsewhere herein. In various embodiments, the medication transfer device 20 may include a user interface 25 for interfacing the user with operations of the device. FIG. 2 illustrates an example of the medication delivery system 10 wherein the medication transfer device 20 includes a dose chamber 40 having measurement markings 42. A user may transfer medication to the dose chamber 40 and use the measurement markings 42 to measure a desired amount of transferred medication. The dose chamber 40 in the illustrated embodiment includes an interior volume 41 defined by rigid sides. However, in other embodiments, a dose chamber may include an interior volume 41 defined by one or more flexible sides and include measurement markings 42. In one example, the transfer module 50 may include a transfer valve that may be selectively opened and closed. For example, the device 20 may include a user interface 25 that interfaces the user with operations of the transfer module 50. The user interface 25 may include manual or automated actuators. For example, a user may press, turn, or slid an actuator to manually open or close a valve or operate a pump. In a further example, a user may provide a control signal at the user interface 25 that causes opening or closing of a valve or operation of a pump. The control signal may result in modulation of power supplied to a solenoid, actuator, or pump, for example. The user interface 25 may include soft buttons, touch screen, digital display, measurement settings, current medication volume, or other system data. In a further example, the transfer module 50 includes a gate valve. Other suitable transfer mechanisms may also be used, such as those described elsewhere herein.

The medication transfer device 20 may comprise various shapes, such a square, rectangular, polygonal, round, or freeform, for example. The dose chamber 40 and the drug chamber 30 may be comprise various relative dimensions and be located at various relative positions. For example, in FIG. 2, the medication transfer device 20 has a rectangular shape with the dose chamber 40 and drug chamber 30 partitioned along the width dimension while FIG. 3 illustrates the medication transfer device 20 where the dose chamber 40 and drug chamber 30 are partitioned along the length dimension.

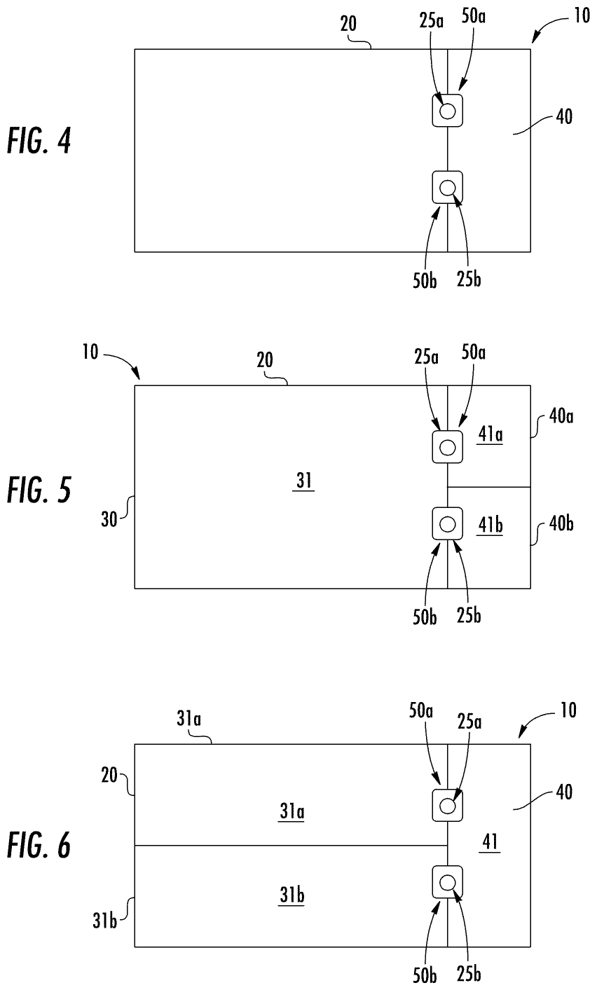

FIG. 4 illustrates a further embodiment of the medication delivery system 10 including a medication transfer device 20 comprising a bag configuration comprising multiple transfer modules 50a, 50b. Multiple transfer modules 50a, 50b may provide dosing flexibility. Each transfer module 50a, 50b may be configured to control transport through a transfer port (e.g., element 51 in FIG. 1) according to any transfer mechanism, such as any transfer mechanisms described herein. In some embodiments, a first transfer module 50a is configured to meter a first volume of medication transferred from the drug chamber 30 to the dose chamber 40 and a second transfer module 50b is configured to transfer a second, different volume of medication from the drug chamber 30 to the dose chamber 40. For example, the medication transfer device 20 may be configured for metering two different volumes of medication utilizing a transfer mechanism that is configured to meter a pre-determined or selected volume between chambers 30, 40. In one embodiment, the second transfer module 50b comprises a one-way valve from the drug chamber 30 to the dose chamber 40 and the first transfer module 50a comprises a one-way valve from the dose chamber 40 to the drug chamber 30. In another embodiment, the first transfer module 50a is configured to transfer or allow transfer of a predetermined or selected predetermined volume, e.g., a metered volume, of medication from the drug chamber 30 to the dose chamber 40 and the second transfer module 50b is configured to allow a user to transfer a desired volume that is not predetermined by the transfer module 50b configuration. For example, the second transfer module 50b may include a transfer valve, such as a shutoff valve, gate valve, compression valve, or stop check-valve, that may be opened to allow transfer and closed to block transfer. Medication may be allowed to flow freely through the opened port or may require motivation, e.g., by squeezing the drug chamber 30 or otherwise increasing pressure within the interior volume 31 of the drug chamber 30 relative to the interior volume 41 of the dose chamber, using a biased spring ball valve, check-valve, duck valve, or a one or two-way pressure valve, as examples.

The medication delivery system 10 including a medication transfer device 20 comprising a bag configuration may also include multiple dose chambers 30 and/or multiple drug chambers 40.

FIG. 5 illustrates an exemplary medication transfer device 20 comprising multiple dose chambers 40a, 40b. A respective transfer module 50a, 50b is included for controlling transfer into each dose chamber 40a, 40b from the drug chamber 30. Each transfer module 50a, 50b may be configured to control transport through a transfer port (e.g., element 51 in FIG. 1) according to any suitable transfer mechanism, such as any transfer mechanisms described herein. In one example, the interior volume 41a, 41b of each the dose chambers 40a, 40b comprises a different volume corresponding to a different volume for transfer, which may comprise all or a portion of a dosage volume. For example, a user may transfer medication sufficient to fill one or the dose chambers 40a, 40b to thereby transfer a measured transfer volume. A medication transfer device 20 comprising multiple dose chambers 40a, 40b each defining a different transfer volume may be utilized to provide multiple dosage volumes that are factors or combinations of the individual transfer volumes defined by the dose chambers 40a, 40b to provide greater dosing flexibility.

FIG. 6 illustrates an exemplary medication transfer device 20 comprising multiple drug chambers 30a, 30b. A respective transfer module 50a, 50b is included for controlling transfer from each drug chamber 30a, 30b to the dose chamber 40. Each transfer module 50a, 50b may be configured to control transport through a transfer port (e.g., element 51 in FIG. 1) according to any suitable transfer mechanism, such as any transfer mechanisms described herein. The transfer modules 50a, 50b may be configured to transfer a same or a different volume of medication, which may be a same or different medication. In one example, the dose chamber 40 comprises a mixing chamber for mixing medication transferred from each of the drug chambers 30a, 30b. It will be appreciated that reference to medication herein with respect to transfer of such may include components or ingredients of a medicinal drug administration. Each transferred component or ingredient need not be a medication. For example, a first drug chamber 30a may contain a pharmaceutical drug and a second drug chamber 30b may contain a base cream for mixing with the pharmaceutical drug. Both transfers may be referred to hearing as transferring of a medication unless indicated otherwise. In some embodiments, multiple drug chambers 40a, 40b may include the same or different pharmaceutical drugs. By maintaining separation of components, instability between such components can be minimized by delaying combination until just prior to administration. Thus, the medication delivery system 10 may be utilized to prevent degradation of unstable ingredients and/or provide multiple measured dosages from a single device.

Other configurations of multiple drug chambers 30, dose chambers 40, and/or transfer modules are also contemplated, for example, a medication delivery system 10 may include a medication transfer device 20 having multiple sets of drug chambers 30, dose chambers 40, and transfer modules 50. The multiple sets may be used to provide different volumes of the same or different medications to be administered together or separate in time and/or location of administration. While multiple user interfaces 25a, 25b are identified in FIGS. 4-6, in some embodiments, a consolidated user interface 25 may be provided to interface the user with the operations of multiple transfer modules 50a, 50b.

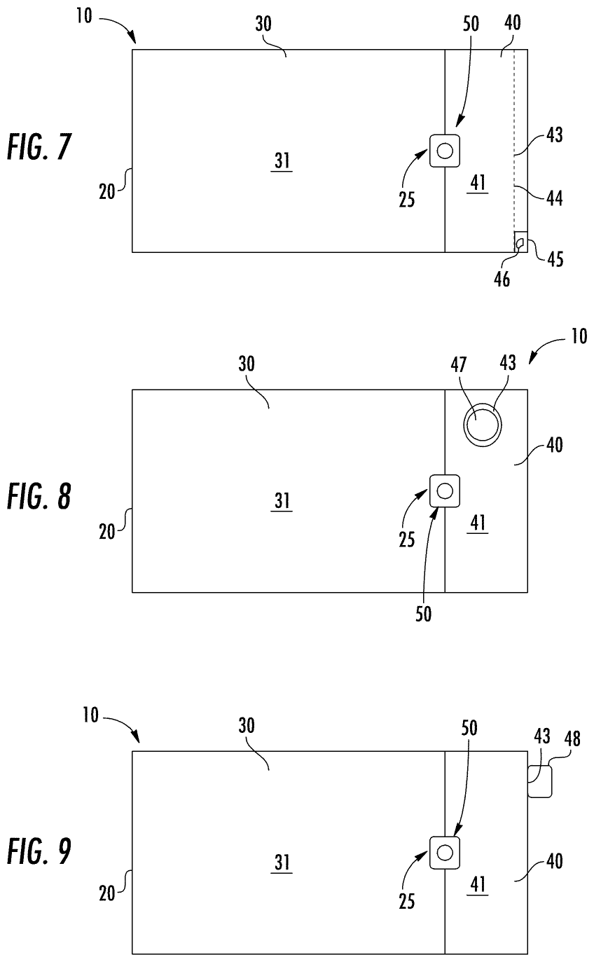

The dose chamber 40 will typically be configured with a dose release port to allow removal of a transfer volume. A dose release port may be configured to be resealable and may include various child-resistant features as described elsewhere herein. FIGS. 7-9 illustrate medication transfer devices 20 comprising exemplary dose release ports 43. In various embodiments, the dose release port 33 has a configuration and associated components as described below and elsewhere herein with respect to a drug port.

The device 20 shown in FIG. 7 depicts a dose release port 43 comprising a resealable zipper 44 formed of mateable flexible zipper halves. The zipper 44 may be sealed and separated by sliding a slider 45 along the zipper 44. In other embodiments, zipper 44 may not include a slider and may be sealed and unsealed by pulling the zipper components apart and compressing the zipper components together, respectively. In one embodiment, the zipper 44 comprises a plastic zipper similar to those found on Ziploc resealable bags. The zipper 44 may include child-resistant features. For example, the slider 45 may be prevented from sliding absent a key. In one embodiment, the slider engages and/or slides beyond biased stops when the dose release port 43 is sealed to prevent sliding of the slider 45 absent removal of the stops or overcoming of the bias, e.g., by physically moving the stops from obstructing the slider 45. In the illustrated embodiment, when the slider 45 seals the dose release port 43, the slider 45 slides over a fixed stop that holds the stop in place. To slide the slider 45 and unseal the zipper 44, pressure must be applied to tab 45 to change the orientation of the slider 45 relative to the stops to allow the slider 45 to slide back over the stops. For example, the slider 45 may include a flexible arm that slides into a recessed stop wherein compressing tab 45 flexes the arm out of the stop to allow the slider 45 to slide back over the stop when compressed.

The device 20 shown in FIG. 8 depicts a dose release port 43 having a cap 47 that threadably engages over the port 43. The cap may include a cap comprising known child-resistant features or other child-resistant features described herein. For example, the cap may comprise a press to turn cap or a squeeze to turn cap.

The device 20 shown in FIG. 9 depicts a dose release port 43 fitted with a valve 48 for sealing or unsealing the dose release port 43. The valve 48 may comprise a gate valve, ball valve, compression valve, or another selectively openable and closable valve. In various embodiments, the valve may be operated by rotating, pressing, or manipulating an actuator.

In some embodiments, an administration mechanism is provided at the dose release port 43. For example, a nozzle for extruding, spraying, sucking, or pouring the transfer volume from the dose release port 43 may be provided.

In various embodiments, the interior volume 31 of the drug chamber 30 is permanently sealed, other than at one or more transfer ports, after medication is received therein to prevent tampering and access.

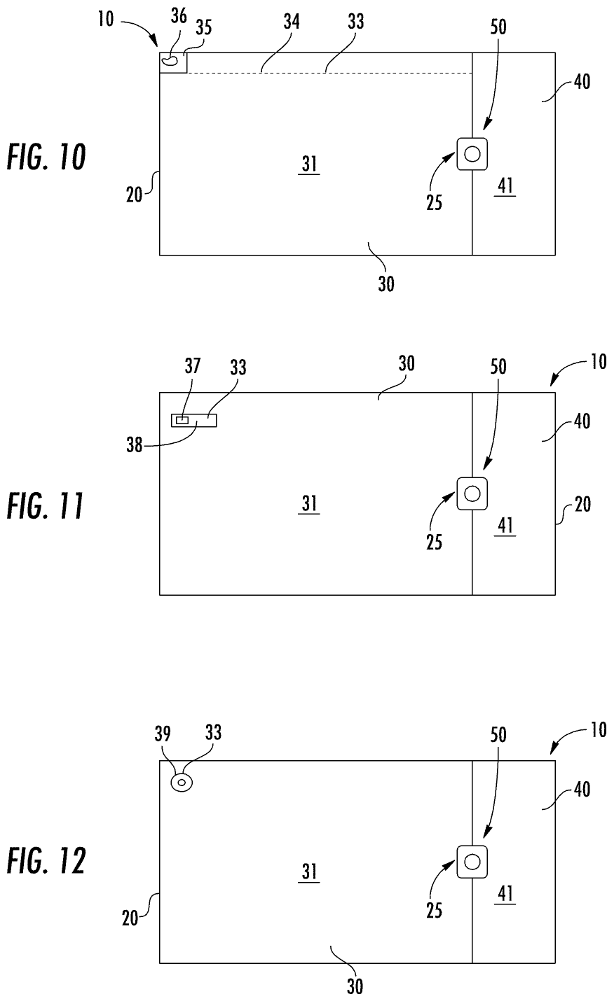

With particular reference to FIGS. 10-12, in some embodiments, the drug chamber 30 may include a fill port 33. The fill port 33 may be used to supply medication into the interior volume 31 of the drug chamber 30, which may be an initial supply or a refill supply. The fill port 33 may have a configuration and associated components as described above and elsewhere herein with respect to the dose release port.

FIG. 10 depicts a medication transfer device 20 including a fill port 33 comprising a resealable zipper 34 formed of mateable flexible zipper halves. The zipper 34 may be sealed and separated by sliding a slider 35 along the zipper 34. In other embodiments, zipper 34 may not include a slider and may be sealed and unsealed by pulling the zipper components apart and compressing the zipper components together, respectively. In one embodiment, the zipper 34 comprises a plastic zipper similar to those found on Ziploc resealable bags. The zipper 34 may include child-resistant features. For example, the slider 35 may be prevented from sliding absent a key. In one embodiment, the slider engages and or slides beyond biased stops when the fill port 33 is sealed that prevents sliding of the slider 35 absent removal of the stops or overcoming of the bias, e.g., by physically moving the stops from obstructing the slider 35. In the illustrated embodiment, when the slider 35 seals the fill port 33, the slider 35 slides over a fixed stop that holds the stop in place. To slide the slider 35 and unseal the zipper 34, pressure must be applied to tab 35 to change the orientation of the slider 35 relative to the stops to allow the slider 35 to slide back over the stops. For example, the slider 35 may include a flexible arm that slides into a recessed stop wherein compressing tab 35 flexes the arm out of the stop to allow the slider 35 to slide back over the stop when compressed.

FIG. 11 depicts a medication transfer device 20 including a fill port 33 comprising a resealable seal. The seal includes a front face 38 and a rear face (not visible) that define the fill port 33. The front face 38 may be flexed outwardly from the rear face to open the fill port 33 and relaxed to engage the rear face for sealing the fill port 33. A lever 37 is provided along the front face 38 for flexing the front face 38. The lever 37 may be moved to relax the front face and operate extensions between the front face 38 and rear face that pull the two faces together along an elastomeric surface therebetween to achieve a seal.

FIG. 12 depicts a medication transfer device 20 including a fill port 33 comprising a valve 39. The valve 39 may comprise sport ball valve configured for a needle adaptor for supplying medication into the drug chamber 30.

In various embodiments, the medication delivery system 10 may include an initial fill or refill container for supplying initial or additional medication into the drug chamber 30. For example, such a container may include a fitting for coupling to the fill port 33 to charge or recharge the drug chamber 30. Fittings may include or couple to a transfer hose. A pump, such as a hand pump may be coupled with the transfer hose. In some embodiments, the container may be flexible for squeezing to generate pressure to drive transfer of medication into the drug chamber 30. In one embodiment, the fill port 33 comprises a Schrader valve and the hose comprises a suitable Schrader valve fitting.

As introduced above, the medication delivery system 10 may include a transfer module 50 for controlling transfer from the drug chamber 30 to the dose chamber 40. The transfer via the transfer module 50 may utilize a variety of transfer mechanisms. For example, transfer valves may be used to control flow, pumps may be utilized to pump fluid, or one or more chambers 30, 40 may be flexible to allow relative pressure to be modulated to drive transfer. In some embodiments, the transfer module 50 may include or utilize a transfer mechanism configured to allow or prevent fluid communication between the interior volumes 31, 41 of the drug chamber 30 and the dose chamber 40. With general reference again to FIG. 1, the transfer module 50 may be configured to allow a user to selectively open and close the transfer port 51. For example, the transfer module 50 may include a shutoff valve operable via manipulation of an actuator. The shutoff valve may include any suitable valve for selectively opening and closing the transfer port 51. It is to be appreciated that opening the transfer port may initiate transfer or allow transfer operations to initiate or be initiated, e.g., via further action of a user, and does not require allowing free flow through the transfer port 51. For example, is some embodiments, opening the transfer port 51 allows free flow of medication through the transfer port, typically from the drug chamber 30 to the dose chamber 40, while in another embodiment, opening the transfer port allows transfer operations of the transfer mechanism to transfer medication. Thus, is one embodiment, opening of a shutoff valve or removal of a barrier to transport allows medication transfer. When transfer is complete, the shutoff valve or other barrier to transport may be closed or activated. Shutoff valves may include, for example, a gate valve, ball valve, or compression valve. Valves may include other barriers such as a movable partition or seal operable to open or close the transfer port.

In some embodiments, a mechanism for selectively opening or closing the transfer port includes a check-valve allowing one-way flow. Check-valves may include a stop check-valve for selectively preventing flow. For example, an actuator may be provided at a user interface 25 to allow a user to manually or via motor assistance engage or disengage the stop function of a stop check-valve.

An actuator with respect to a valve, e.g., shutoff valve or stop check-valve, may comprise a switch, push button, knob, or a touch screen button of a user interface, for example. The pushing, pulling, twisting, sliding, switching, or otherwise actuating the actuator may cause opening or closing of the transfer port 51. Actuation may cause a physical barrier to obstruct or reveal the transfer port 51. In some embodiments, actuation may initiate or drive transfer of medication from the drug chamber 30 to the dose chamber 40 through the transfer port 51. In one embodiment, actuation may cause a measurement component of the transfer module 50 to pump in or receive medication from the interior volume 31 of the drug chamber 30 and/or to pump out or release medication into the interior volume 41 of the dose chamber 40, which may comprise a measured portion of medication.

As introduced above, a mechanism for selectively opening and closing the transfer port 51, or allowing or preventing transfer, may be operated manually, electronically, and/or via motor assistance, which may be automated, e.g., operation of an switch, button, or actuator corresponding to initiation of medication transfer, or otherwise selectable by a user, e.g., operation of a switch, button, or actuator corresponding to powering a motor configured to open or close a transfer valve or manipulate a barrier or structure to open or close the transfer port 51.

FIG. 13 illustrates a magnified view of a transfer module 50 comprising an actuator 52 for actuating a transfer valve, such as a shutoff valve, stop check-valve, or pressure activated check-valve (not shown). The actuator 52 illustrated comprises a knob. The knob may be rotatable in directions indicated by arrows B and C to open and close the valve. In this or another embodiment, the actuator 52 may be pulled or pushed to open or close the valve. When open, the valve may be pressure operated such that a threshold pressure must be provided for flow. In another example, the valve is not configured for requiring additional pressure for transfer when open.

In some embodiments, the actuator 52 may be pressed to open and close the transfer valve, which may comprise a stop check-valve, and further rotated to initiate or drive transfer of medication. In one embodiment, the actuator 52 may be rotated to open and close the valve and pressed to initiate or drive transfer of medication. In one embodiment, actuating the actuator 52 drives or initiates rotation or movement of a metering wheel, movement of a plunger, and/or movement of a transfer chamber. For example, the actuator 52 may be rotated to a position corresponding to a desired predetermined transfer volume. Rotation may cause a corresponding rotation of a metering wheel. The degree of rotation may or may not correspond to a degree of rotation of the actuator. For example, rotation may initiate a motor for providing a particular number of rotations of the metering wheel or may actuate a biasing structure such as a spring configured to cause a desired number of rotations of the metering wheel. It is noted that other metering structures may be used and need not have a ball or wheel shape. For example, actuation may cause movement of a transfer chamber or a plunger within a transfer chamber or in fluid communication therewith.

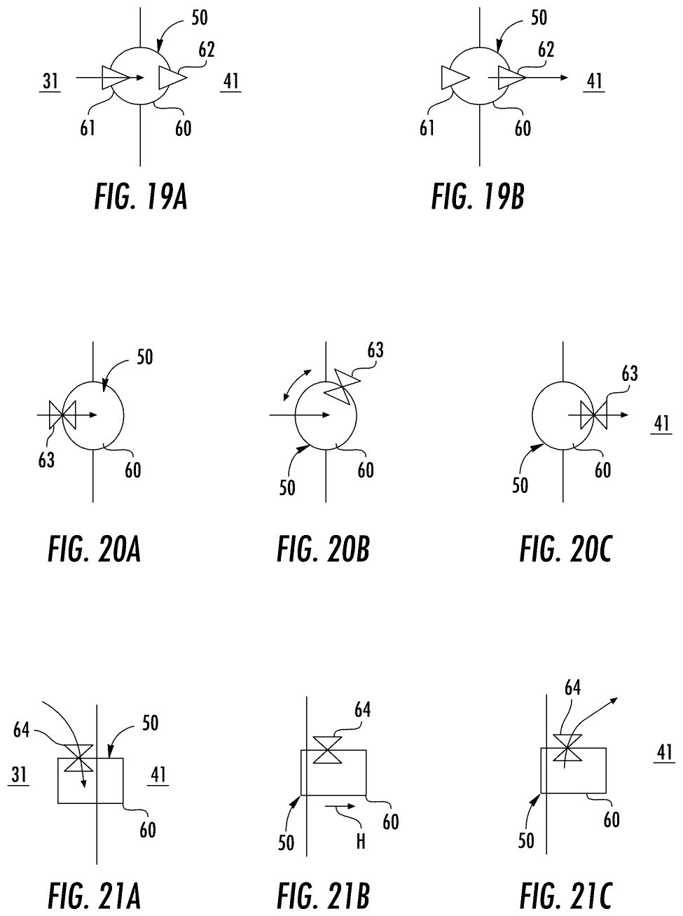

In one embodiment, the actuator 52 may be rotated or pulled outwardly to actuate a plunger to fill a transfer chamber (not shown) with medication from the interior volume 31 of the drug chamber 30 and then rotated or pushed in to pump the medication from the transfer chamber into the interior volume 41 of the dose chamber 40, which may include driving the plunger back through the transfer chamber. In one such example, actuation causing movement of the plunger also causes sequenced opening of the transfer port, which may comprise multiple transfer ports, in communication with the transfer chamber. In some embodiments, the transfer chamber includes a transfer valve arrangement similar to that described with respect to FIG. 19A & 19B comprising a one-way valve for receiving medication from the interior volume 31 of the drug chamber 30 when the plunger decreases pressure within the transfer chamber below that within the interior volume 31 of the drug chamber 30 and a one-way valve for delivering medication into the interior volume 41 of the dose chamber 40 when the plunger increases pressure within the transfer chamber above that within the interior volume 41 of the drug chamber. In some embodiments, rotating, pulling or pushing actuator 52 initiates a motor for providing a particular number of rotations of the metering wheel, length or number of strokes of a plunger through a transfer chamber, or may actuate a biasing structure such as a spring configured to cause a desired movements of the metering wheel or plunger. In some embodiments, the actuator 52 may be configured with child-resistant features, such as those described herein.

FIG. 14 illustrates a magnified view of a transfer module 50 comprising a check-valve 53 for allowing one-way transfer from the interior volume 31 of the drug chamber 30 to the interior volume 41 of the dose chamber 40. The check-valve 53 may comprise a duck valve, stop check-valve, ball valve, or other suitable valve for allowing one-way transfer. As noted above, the check-valve 53 may comprise a pressure activated check-valve.

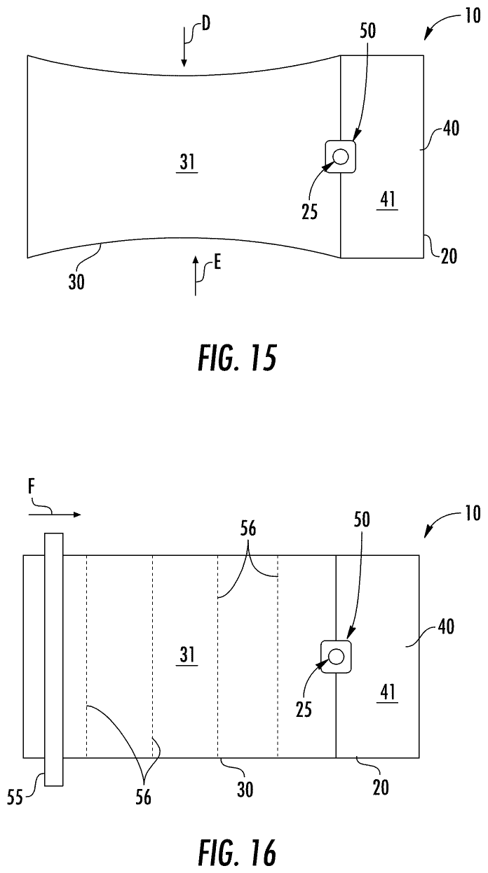

As introduced above, the transfer mechanism may include increasing pressure within the interior volume 31 of the drug chamber 30. For example, FIG. 15 illustrates another embodiment of the medication delivery system 10 wherein the transfer mechanism includes flexible walls of the drug chamber 30 wherein compressing the interior volume 31, as indicated by arrows D, E, increases pressure within the interior volume 31 to drive transfer of medication. The transfer module 50 may be operable to control transfer through the transfer port and/or transfer volume and may comprise one or more transfer valves, metering structures such as wheels or chambers, or other suitable components, such as those described here.

In some embodiments, the transfer mechanism includes a movable partition. For example, FIG. 16 illustrates another embodiment of the medication delivery system 10 wherein the transfer mechanism includes a partition 55 that is slidable along arrow F to partition the drug chamber 30. The partition 55 may extend within the interior volume 31, compress the interior volume externally, or both. The drug chamber 30 may also include measurement markings corresponding to a transfer volume, dosage volume, and/or standard measurements. A user may slide the partition 55 in direction of arrow F to the desired measurement marking and thereby decrease the interior volume 31, increase pressure, and drive transfer of the medication. The transfer module 50 may be operable to control transfer through the transfer port and/or transfer volume and may comprise one or more transfer valves, metering structures such as wheels or chambers, or other suitable components, such as those described here.

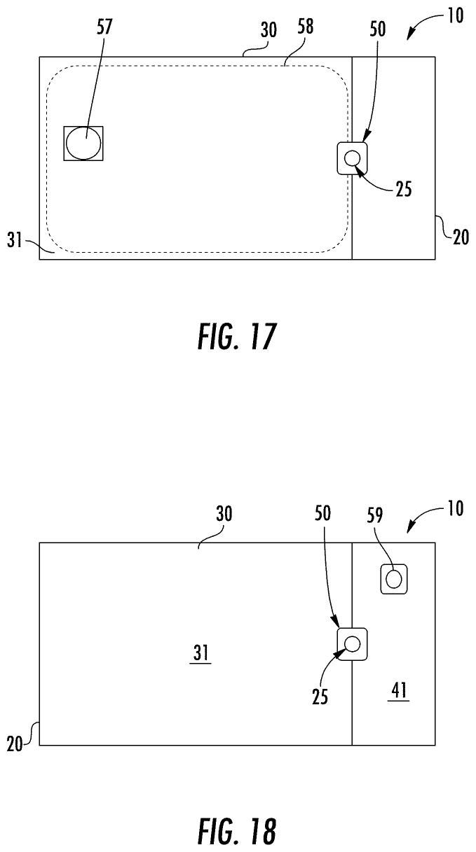

FIGS. 17 & 18 illustrates additional transfer mechanisms that modulate pressure to drive transfer. In FIG. 17, a pressure actuator 57 is provided along the drug chamber 30. The pressure actuator 57 may comprise an inflatable bladder 58 that may be inflated by actuation of the pressure actuator 57. In one example, a user may measure a desired transfer volume by actuating the pressure actuator 57 a predetermined number of times. That is, an actuation of the pressure actuator 57 may correspond to a predetermined amount of medication transferred and a user may actuate the pressure actuator 57 a number of times corresponding to their dosage volume to ensure the proper amount of medication is transferred. In FIG. 18, the dose chamber 40 is equipped with a pressure actuator 59 configured to decrease pressure within the interior volume 41 of the dose chamber 40 relative to the drug chamber 30. The pressure actuator 59 may include a diaphragm that may be pulled to decrease pressure or an extension that may be extended within the interior volume 41 to separate sides of the dose chamber 40. In some embodiments, pressure actuators 58, 59 may be provided at a user interface 25.

FIGS. 19A & 19B semi-schematically illustrate operation of a transfer module 50 configured for use with a transfer mechanism comprising a transfer chamber 60 and two valves 61, 62. The valves 61, 62 are positionable in the separate interior volumes 31, 41. The valves are preferably one-way valves comprising stop check-valves or other check-valves. In some embodiments, the valves 61, 62 may comprise pressure activated check-valves. It will be appreciated that in some instances valves 61, 62 may include two-way valves with selectable flow direction or shutoff valves operated to achieve the effect of one-way valves within the transfer operation. The direction of flow is from interior volume 31 into the transfer chamber 60 in FIG. 19A and into interior volume 41 from the transfer chamber 60 in FIG. 19B.

In one embodiment, the transfer chamber 60 comprises a volume that may be decreased and subsequently returned to a previous volume. For example, the transfer chamber 60 may include one or more flexible walls that may be flexed to decrease the volume of the transfer chamber 60. The transfer chamber 60 may include a movable partition for drawing in and pushing out medication. In one embodiment, the transfer chamber 60 includes a flexible bladder (e.g., a balloon). The bladder may be compressed by manipulation, e.g., utilizing an actuator or directly with the hand of a user. Depressing a side of the bladder drives a reduction in volume within the transfer chamber 60. As the transfer chamber 60 returns to a pre-compressed volume, medication is drawn into the transfer chamber 60. Subsequent compression of the volume may drive delivery of the medication from the transfer chamber 60 into the interior volume 41 of the drug chamber. Thus, in such a configuration, a predetermined amount of medication transferred may be associated with each actuation or depression, e.g., pump, of the bladder and the number of pumps may be used to determine the appropriate transfer volume. While the transfer chamber is illustrated as having a round cross-section, in various embodiments, the transfer chamber 60 may comprise a different cross-section such as oblong, square, triangular, rectangular, polygonal, or freeform.

In one embodiment, the transfer mechanism includes an actuator, which may be similar to that shown in FIG. 13 for example, that is coupled to a diaphragm. Rotation of the actuator extends and retracts the diaphragm in a precise manner to pull medication into the transfer chamber 60 from the interior volume 31 of the drug chamber (FIG. 19A) and push medication out of the transfer chamber 60 into the interior volume 41 of the dose chamber (FIG. 19B). The number of rotations may be used to set a predetermined transfer volume. In a similar configuration, an actuator may be pulled and pushed to manipulate the diaphragm. In another example, the actuator may be coupled to a plunger wherein actuation of the plunger reduces pressure in transfer chamber 60 relative to the interior volume 31 of the drug chamber to pull medication into the transfer chamber 60 from the interior volume 31 of the drug chamber (FIG. 19A) and a counter actuation works to increase the pressure within the transfer chamber 60 relative to the interior volume to push medication out of the transfer chamber 60 into the interior volume 41 of the dose chamber (FIG. 19B). In some embodiments, pressure differentials to drive medication flow may be provided as described with respect to FIGS. 15-18. As noted above, actuation may be automated or assisted by a motor. In one embodiment, a user interface is provided allowing a user to enter a desired volume or actuations and a controller processes the request and executes the appropriate actuations to achieve the volume.

FIGS. 20A-20C semi-schematically illustrate operation of a transfer module 50 configured for use with a transfer mechanism comprising a transfer chamber 60 and one or more valves 63 arranged to operate as a two-way valve. The one or more valves 63 may be pressure activated in one or both directions. The transfer chamber 60 or at least one of the one or more valves 63 is configured to be movable to provide an outlet in fluid communication with interior volume 41 and/or interior volume 31. In the illustrated embodiment, the transfer chamber 60 is movable along arrow G, as indicated in FIG. 20B, to selectively position the one or more valves 63 into fluid communication with interior volume 31 and interior volume 41. In other embodiments, the transfer chamber 60 or one or more valves 63 may be movable to a greater or lesser degree or may be movable in only a single direction. The one or more valves 63 may comprise shutoff valves that may be selectively opened and closed. In one embodiment, pressure activation corresponds to pressure provided by an actuation of a plunger. In another embodiment, the transfer chamber 60 is compressible and pressure activation corresponds to pressure provided by compression of transfer chamber 60. The direction of flow is from interior volume 31 into the transfer chamber 60 in FIG. 20A and into interior volume 41 from the transfer chamber 60 in FIG. 20C. The transfer chamber 60 is rotatable to position the two-way valve

In one embodiment, the transfer chamber 60 comprises a volume that may be decreased and subsequently returned to a previous volume. For example, the transfer chamber 60 may include one or more flexible walls that may be flexed to decrease the volume of the transfer chamber 60. The transfer chamber 60 may include a movable partition for drawing in and pushing out medication. In one embodiment, the transfer chamber 60 includes a flexible bladder (e.g., a balloon). The bladder may be compressed by manipulation, e.g., utilizing an actuator or directly with the hand of a user. Depressing a side of the bladder drives a reduction in volume within the transfer chamber 60. As the transfer chamber 60 returns to a pre-compressed volume, medication into taken into the transfer chamber 60 (FIG. 20A). The transfer chamber 60 may then be rotated to position at least one of the one or more valves 63 in fluid communication with the interior volume 41 of the dose chamber (FIG. 20B). Subsequent compression of the volume of the transfer chamber 60 may drive delivery of the medication from the transfer chamber 60 into the interior volume 41 of the drug chamber (FIG. 20C). Thus, in such a configuration, a predetermined amount of medication transferred may be associated with each actuation or depression, e.g., pump, of the bladder and the number of pumps together with the number of turns to release the medication from the transfer chamber 60 may be used to determine the appropriate transfer volume. While the transfer chamber is illustrated as having a round cross-section, in various embodiments, the transfer chamber 60 may comprise a different cross-section such as oblong, square, triangular, rectangular, polygonal, or freeform.

In one embodiment, the transfer mechanism includes an actuator, which may be similar to that shown in FIG. 13 for example, that is coupled to a diaphragm. Rotation of the actuator extends and retracts the diaphragm in a precise manner to pull medication into the transfer chamber 60 from the interior volume 31 of the drug chamber (FIG. 20A) and push medication out of the transfer chamber 60 into the interior volume 41 of the dose chamber (FIG. 20C). The number of rotations may be used to set a predetermined transfer volume. In a similar configuration, an actuator may be pulled and pushed to manipulate the diaphragm. In another example, the actuator may be coupled to a plunger wherein actuation of the plunger reduces pressure in transfer chamber 60 relative to the interior volume 31 of the drug chamber to pull medication into the transfer chamber 60 from the interior volume 31 of the drug chamber (FIG. 20A) and movement of the transfer chamber 60 (FIG. 20B) followed by a counter actuation works to increase the pressure within the transfer chamber 60 relative to the interior volume 41 to push medication out of the transfer chamber 60 into the interior volume 41 of the dose chamber (FIG. 20C). In some embodiments, pressure differentials to drive medication flow may be provided as described with respect to FIGS. 15-18. As noted above, actuation may be automated or assisted by a motor. In one embodiment, a user interface is provided allowing a user to enter a desired volume or actuations and a controller processes the request and executes the appropriate actuations to achieve the volume.

FIGS. 21A-21C semi-schematically illustrate operation of a transfer module 50 configured for use with a transfer mechanism comprising a transfer chamber 60 and one or more valves 64 arranged to operate as a two-way valve. The one or more valves 64 may be pressure activated in one or both directions. The transfer chamber 60 or at least one of the one or more valves 64 is configured to be movable to provide an outlet in fluid communication with interior volume 41 and/or interior volume 31. In the illustrated embodiment, the transfer chamber 60 is movable along arrow H, as indicated in FIG. 21B, to selectively position the one or more valves 64 into fluid communication with interior volume 31 and interior volume 41. In other embodiments, the transfer chamber 60 or one or more valves 64 may be movable to a greater or lesser degree or may be movable in only a single direction. The one or more valves 64 may comprise shutoff valves that may be selectively opened and closed. In one embodiment, pressure activation corresponds to pressure provided by an actuation of a plunger. In another embodiment, the transfer chamber 60 is compressible and pressure activation corresponds to pressure provided by compression of transfer chamber 60. The direction of flow is from interior volume 31 into the transfer chamber 60 in FIG. 21A and into interior volume 41 from the transfer chamber 60 in FIG. 21C. The transfer chamber 60 is rotatable to position the two-way valve

In one embodiment, the transfer chamber 60 comprises a volume that may be decreased and subsequently returned to a previous volume. For example, the transfer chamber 60 may include one or more flexible walls that may be flexed to decrease the volume of the transfer chamber 60. The transfer chamber 60 may include a movable partition for drawing in and pushing out medication. In one embodiment, the transfer chamber 60 includes a flexible bladder (e.g., a balloon). The bladder may be compressed by manipulation, e.g., utilizing an actuator or directly with the hand of a user. Depressing a side of the bladder drives a reduction in volume within the transfer chamber 60. As the transfer chamber 60 returns to a pre-compressed volume, medication is drawn into the transfer chamber 60 (FIG. 21A). The transfer chamber 60 may then be rotated to position at least one of the one or more valves 63 in fluid communication with the interior volume 41 of the dose chamber (FIG. 21B). Subsequent compression of the volume of the transfer chamber 60 may drive delivery of the medication from the transfer chamber 60 into the interior volume 41 of the drug chamber (FIG. 21C). Thus, in such a configuration, a predetermined amount of medication transferred may be associated with each actuation or depression, e.g., pump, of the bladder and the number of pumps together with the number of turns to release the medication from the transfer chamber 60 may be used to determine the appropriate transfer volume. While the transfer chamber is illustrated as having a rectangular cross-section, in various embodiments, the transfer chamber 60 may comprise a different cross-section such as round, oblong, square, triangular, polygonal, or freeform.

In one embodiment, the transfer mechanism includes an actuator, which may be similar to that shown in FIG. 13 for example, that is coupled to a diaphragm. Rotation of the actuator extends and retracts the diaphragm in a precise manner to pull medication into the transfer chamber 60 from the interior volume 31 of the drug chamber (FIG. 21A) and push medication out of the transfer chamber 60 into the interior volume 41 of the dose chamber (FIG. 21C). The number of rotations may be used to set a predetermined transfer volume. In a similar configuration, an actuator may be pulled and pushed to manipulate the diaphragm. In another example, the actuator may be coupled to a plunger wherein actuation of the plunger reduces pressure in transfer chamber 60 relative to the interior volume 31 of the drug chamber to pull medication into the transfer chamber 60 from the interior volume 31 of the drug chamber (FIG. 21A) and movement of transfer chamber 60 (FIG. 21B) followed by a counter actuation works to increase the pressure within the transfer chamber 60 relative to the interior volume 41 to push medication out of the transfer chamber 60 into the interior volume 41 of the dose chamber (FIG. 21C). In some embodiments, pressure differentials to drive medication flow may be provided as described with respect to FIGS. 15-18. As noted above, actuation may be automated or assisted by a motor. In one embodiment, a user interface is provided allowing a user to enter a desired volume or actuations and a controller processes the request and executes the appropriate actuations to achieve the volume.

While the features of the medication delivery system 10 described with respect to FIGS. 1-21C have been described with respect to a medication transfer device 20 having a bag configuration, such features are also contemplated for utilization in other medication transfer device configurations such as pouches, bottles, jars, and other configurations, such as those described elsewhere herein.

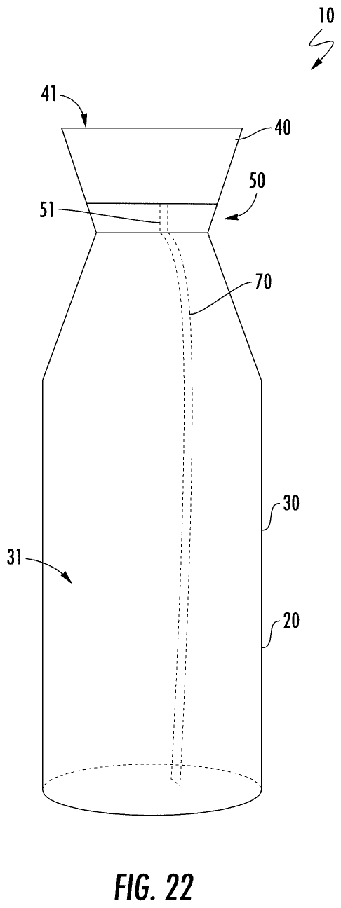

FIGS. 22-29 illustrate various embodiments of the medication delivery system 10 wherein the medication transfer device 20 comprises bottle configuration. With reference to FIG. 22, the medication transfer device 20 includes a drug chamber 30, dose chamber 40, and transfer module 50 for providing transfer of medication through a transfer port 51 from the interior volume 31 of the drug chamber 30 to the interior volume 41 of the dose chamber 40. In the illustrated embodiment, a hose 70 extends through the interior volume 31 of the drug chamber to interface with the transfer module 50. The chambers 30, 40 may comprise generally liquid impermeable material such as glass; plastic such as PVC, vinyl, polypropylenes (PP), polyethylenes (PE), polyethylene terephthalates (PET) such as boPET, or other plastics; ceramic; metal; or combinations thereof.

The medication transfer device 20 is configured to contain multiple dosages of medication within the drug chamber 30 and is operable to transfer a transfer volume of the medication into the interior volume 41 of the dose chamber 40. The transfer volume may be a predetermined, selectable, or customizable volume of medication according to a configuration of the transfer module 50 or device configuration, as described in more detail elsewhere herein. In various embodiments, the transfer module 50 may include a transfer valve (not shown) that may be selectively opened and closed, e.g., via user interaction with a user interface 25 or by assembling the device 20. In a further example, the transfer module 50 includes a gate valve. Control of the transfer valve may be used to prevent unwanted initiation of the transfer mechanism leading to unwanted transfer of medication and tampering by children. The medication transfer device 20 may be configured to utilize any suitable transfer mechanism, such as those described herein. The transfer module 50 may comprise a manual pump or a pump that couples to the output of a motor configured for assisted or automated pumping. In various embodiments, the transfer module 50 includes a valve configuration, bladder, and/or transfer chamber as described above with respect to FIGS. 19A-21C.

The transfer module 50 may include a valve such as a shutoff valve or stop check-valve. In some embodiments, users may interface with operations of the valve at a user interface 25. The user interface 25 may comprise one or more switches, push buttons, knobs, or soft buttons, for example. The user interface 25 may include or operatively couple with, e.g., be operable to control, an actuator for opening and/or closing the valve. In one embodiment, the user interface 25 includes a control component that may be pushed, pulled, rotated, slid, switched, or otherwise actuated to cause opening or closing of the transfer port 51 (see FIG. 22). Actuation may cause a physical barrier to obstruct or reveal the transfer port 51. In some embodiments, initiation of transfer operations at the user interface 25 may open the valve for transfer operations. For example, initiation of transfer operations may not require additional steps that cause opening and closing of the valve. In one example, a user interface 25 may include button that initiates a pump to transfer medication. The pump may be electrically powered and/or manually powder, e.g., depressing the button may compress a bladder or bulb for hand pumping. However, in some embodiments, the transfer module 50 may maintain closure of the valve until a key, code, button sequence, or child-resistant feature has been overcome. As described in more detail below, in some embodiments, child-resistant features may include installation of the dose chamber 40, transfer module 50, or both. The user interface 25 may be distributed to locations other than the transfer module 50.

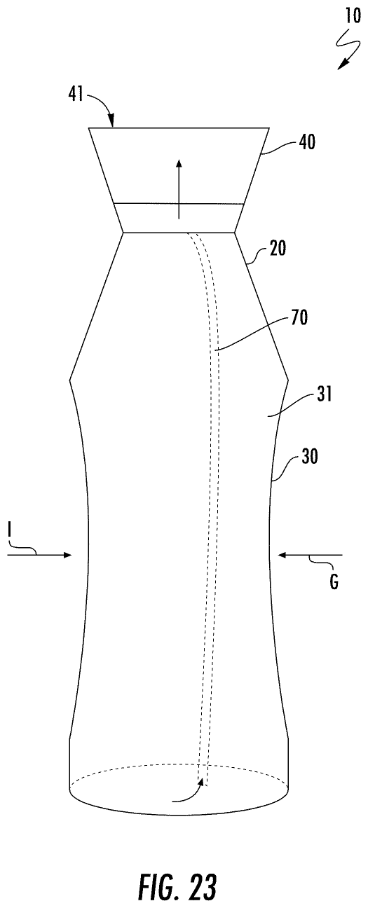

FIG. 23 illustrates an embodiment of the medication delivery system 10 wherein the medication transfer device 20 comprises a drug chamber 30 having a compressible interior volume 31. For example, one or more sides of the drug chamber 30 may be flexible to allow a user to compress the side inwardly, as indicated by arrows I, G, to reduce the interior volume 31. Reduction in the interior volume 31 drives flow of medication within the interior volume 31 through the hose 70 to the transfer module 50. The transfer module 50 may include a transfer valve, such as a shutoff valve or stop check-valve operable via an actuator 26 comprising a switch, push button, knob, or a touch screen button of a user interface, for example. Pushing, pulling, twisting, sliding, switching, or otherwise actuating the actuator 26 may cause opening or closing of the transfer port 51 (see FIG. 22). Actuation may cause a physical barrier to obstruct or reveal the transfer port 51. In some embodiments, the transfer module 50 may maintain closure of the valve until a key, code, or child-resistant feature has been overcome. As described in more detail below, in some embodiments, child-resistant features may include installation of the dose chamber 40, transfer module 50, or both. A button or switch may be provided by the transfer module 50 that may be actuated to open a transfer valve when the dose chamber 40 and/or transfer module 50 have been coupled.

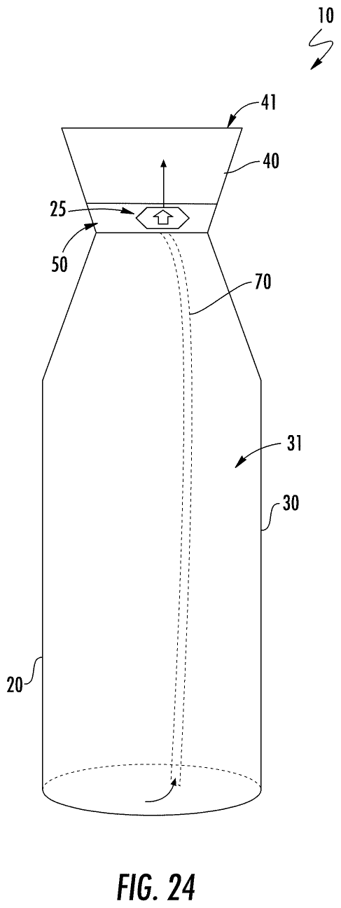

With reference to FIG. 24, in some embodiments, the user interface 25 may include a button or other control component, which is generally referred to as an actuator 26 herein, wherein actuation of the actuator 26 initiates or drives transfer of medication from the drug chamber 30 to the dose chamber 40 through the transfer port. The actuator 26 may directly control operations by physically translating actuation to move a pump, move a transfer chamber, or translate valves, for example. In some embodiments, the actuator 26 may provide control instructions via an electrical circuit to control power to components such as an electric pump or solenoid. In further embodiments, the actuator 26 may provide control instructions to a control system processor that processes the instruction and executes a corresponding action based on programmed software. In one embodiment, actuation may cause a measurement component of the transfer module 50 to pump in or receive medication from the interior volume 31 of the drug chamber 30 and/or to pump out or release medication into the interior volume 41 of the dose chamber 40, which may comprise a measured portion of medication. Actuator 26 may be actuated to initiate a powering of a pump during actuation or for a predetermined time following actuation, e.g., based on a transfer volume entered by the user at the user interface 26. In one example, the actuator 26 or another user interface control component may be manipulated to specify a desired transfer volume that is used to determine pumping time. In one embodiment, actuator 26 may comprise a manual pump whereby actuation drives pumping of medication.

As introduced above, a mechanism for selectively opening and closing the transfer port 51, or allowing or preventing transfer, may be operated manually, electronically, and/or via motor assistance, which may be automated, e.g., operation of an switch, button, or actuator corresponding to initiation of medication transfer, or otherwise selectable operation by a user, e.g., operation of a switch, button, or actuator corresponding to powering a motor configured to open or close a valve or manipulate a barrier or structure to open or close the transfer port.

In various embodiments, the transfer module 50 comprises a control system including a processor configured to execute pumping operations to provide precise transfer and/or dosage volumes. For example, the control system may include a user interface 25 comprising a touch screen, one or more hard or soft buttons, knobs, switches, or other control components, which may comprise actuators 26 as noted above. The control system may include computer readable memory storing instructions that when executed by the processor execute operations of the control system. The control system may be operable to control operations of a motor and pump for pumping medication into the dose chamber 40. In one embodiment, the control system includes one or more sensors such as depth sensors, flow rate sensors, pressure sensors, weight sensors, optical sensors, or other sensors configured for determining a transfer volume. A user may enter a desired transfer or dosage volume into the user interface. The control system may then control operations of the pump to provide the entered volume.

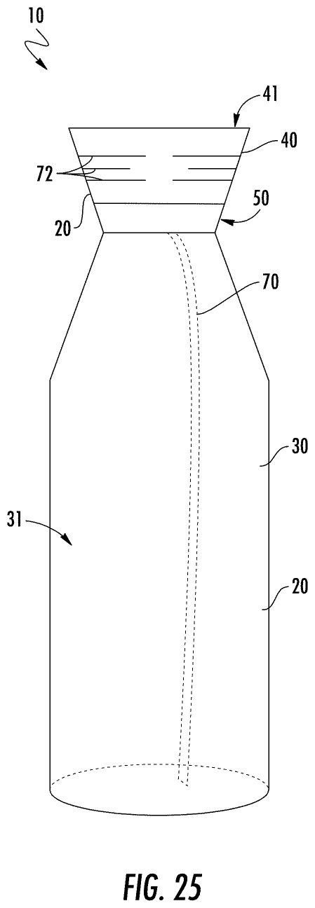

FIG. 25 illustrates an example of the medication delivery system 10 wherein the medication transfer device 20 includes a dose chamber 40 having measurement markings 42. A user may transfer medication to the dose chamber 40 and use the measurement markings 42 to measure a desired amount of transferred medication. The dose chamber 40 in the illustrated embodiment includes an interior volume 41 defined by rigid sides. However, in other embodiments, a dose chamber may include an interior volume 41 defined by one or more flexible sides (see, e.g., FIG. 23) and include measurement markings 42.

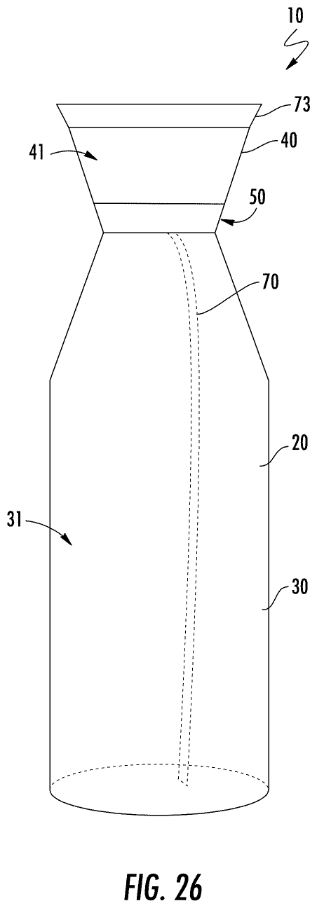

FIG. 26 illustrates an example of a medication transfer device 20 having a lid 73 for covering the dose chamber 40. The lid 73 may include a fitting for coupling with a complementary fitting disposed on the dose chamber 40. In various embodiments, the lid 73 coupling with the dose chamber includes a child-resistant features configured to resist decoupling by children, e.g., in compliance with the Poison Prevention Packaging Act or as otherwise with the Federal Drug Administration or U.S. Consumer Product Safety Commission. For example, the decoupling may require local squeeze force applied while turning, holding fitment while turning, pushing down while turning, pushing down into limited fitment gap followed by turning to secondary fitment gap, aligning cap and ring followed by pushing cap toward ring and turning, localized pushing up to remove, localized pressing down then pulling up at arrow, turning cap to stop and then lifting and continuing opening, localized pushing in while turning where force must be applied to designated place on closure, or other child-resistance mechanisms, which may include others described herein. Additional child-resistant features may include lug finish closures or continuous threads.

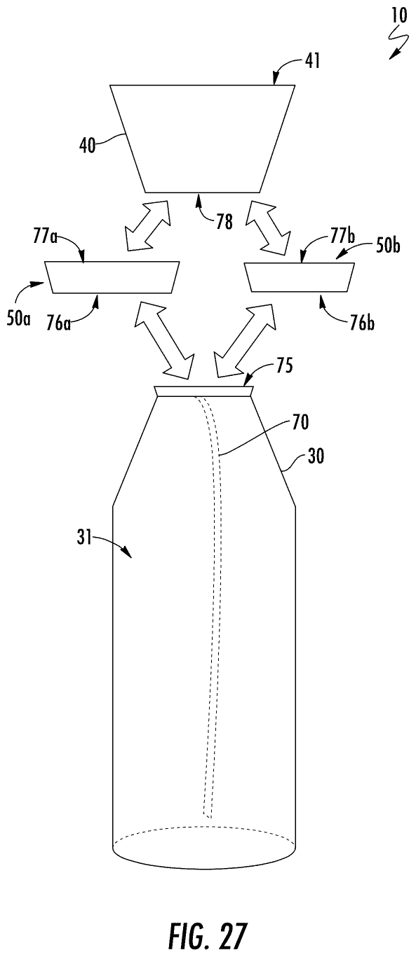

One or more components of the medication delivery system 10 may comprise modular and/or interchangeable components. With reference to FIG. 27, a medication delivery system 10 may comprise modular assembly of a drug chamber 30, dose chamber 40, and transfer module 50a, 50b. As shown, multiple interchangeable transfer modules 50a, 50b may be provided. In some embodiments, transfer modules 50a, 50b may be configured to transfer specific or specific ranges of transfer volumes. Users may be provided one or more suitable transfer modules 50a, 50b for the transfer volume needed. In some embodiments, a method of providing a medication treatment comprises providing a transfer module 50a, 50b configured to provide an appropriate transfer volume for a prescribed dosage. In this or another embodiment, a method of providing a medication treatment may comprise providing a drug chamber 30 as described herein containing multiple dosages of a prescribed medication treatment. The drug chamber 30 may be sealed as to not spill or become contaminated during shipping or delivery. The drug chamber 30 may include a fitting 75 configured to couple to a corresponding fitting 76a, 76b of a transfer module 50a, 50b, which may include fittings disposed on a plurality of interchangeable transfer modules 50a, 50b. A drug port (not visible) may be positioned near fitting 75, around the area of coupling, for alignment and coupling with the transfer port 51. The drug port and transfer port 51 may align when coupled. In some embodiments, the hose 70 couples to a closure valve (not visible) at or around the fitting 75. The fitting 76a, 76b of the transfer module 50a, 50b may engage or associate with the closure valve, hose 70, or extension thereof. In some embodiments, the engagement or association may result in providing control of the operation of closure valve to the transfer module 50a, 50b, e.g., via operation of an actuator. In the illustrated embodiment, coupling the fitting 75 to fitting 76a, 76b does not open the closure valve; however, in other embodiments, coupling the fittings 75, 76a, 76b opens the closure valve. In one such example, the transfer module 50a, 50b seals or blocks the transfer port utilizing a valve, e.g., as described herein, to prevent undesirable transfer of medication until initiation of transfer. Dose chamber 40 may include a fitting 78 configured to couple to a complementary fitting 77a, 77b of the transfer module 50a, 50b, which may include fittings disposed on a plurality of interchangeable transfer modules 50a, 50b. Coupling the dose chamber 40 and transfer module 50a, 50b may align a port defined in the fitting 78 of the dose chamber 40 with the transfer port extending through the transfer module 50a, 50b. In various embodiments, the transfer module 50a, 50b is prevented for opening the transfer port or closure valve unless properly coupled to the drug chamber 30, dose chamber 40, or both. Thus, in one example, operation of a transfer actuator or otherwise initiating transfer operations at the user interface 25 may be prevented if the transfer module 50a, 50b is coupled to the drug chamber 30 but not properly coupled to the dose chamber 40.

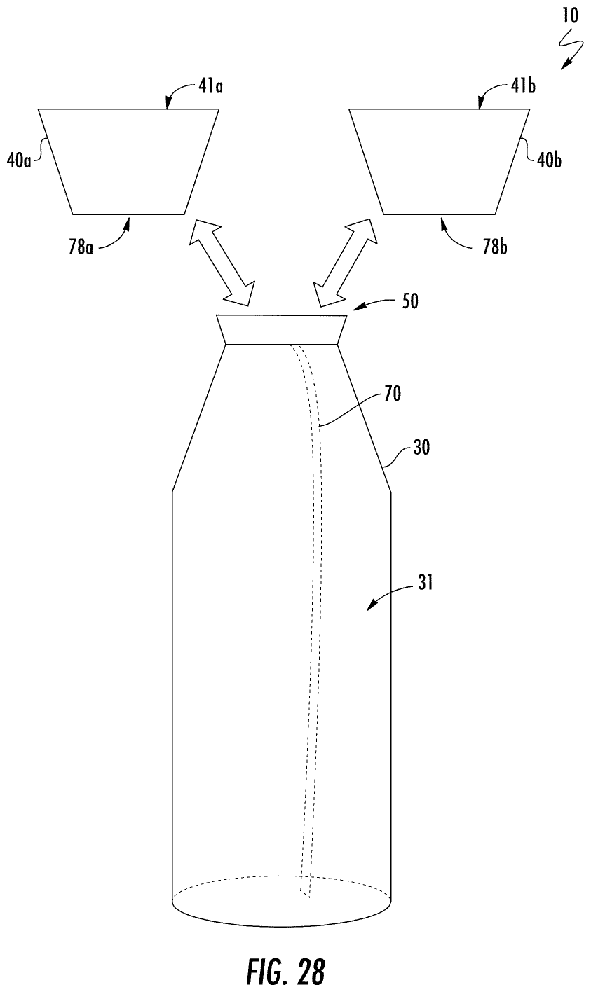

FIG. 28 illustrates a further example of a modular medication delivery system 10 including modular dose chambers 40a, 40b. The medication delivery system 10 may also comprise interchangeable dose chambers 40a, 40b. The dose chambers 40a, 40b may comprise the same or different interior volumes 41a, 41b. The dose chambers 40a, 40b may each comprise a fitting 78a, 78b configured to couple to a corresponding fitting 77 disposed on the transfer module 50 as described above with respect to FIG. 27. The drug chamber 30 may be sealed as to not spill or become contaminated during shipping or delivery. Transfer operations may similarly be prevented until the transfer module 50 is properly coupled to the dose chamber 40. In various embodiments, users may be provided drug chambers containing medication and a transfer module 50 for transferring a transfer dose corresponding to a prescribed dosage. The user may couple the dose chamber 40 and transfer module 50 to transfer and thereby dispense the medication from the drug chamber 30.

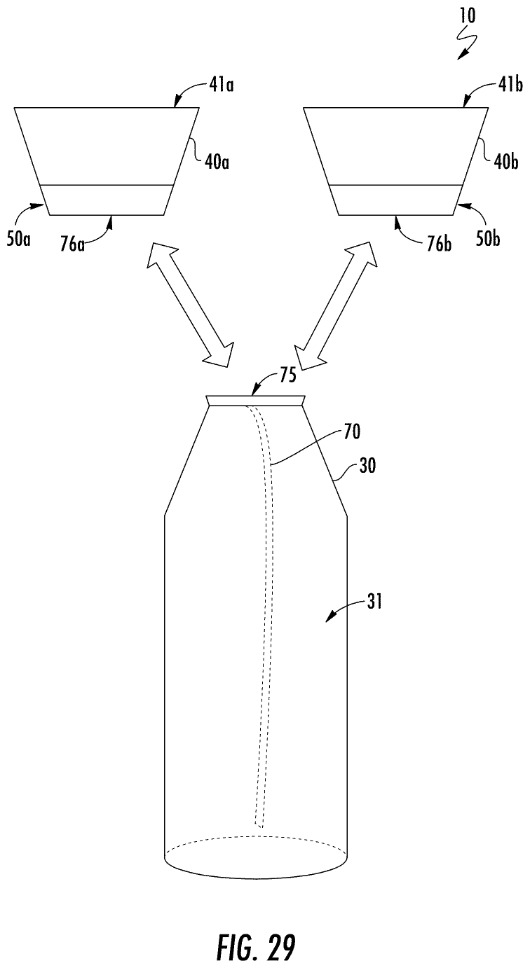

FIG. 29 illustrates a further example of a modular medication delivery system 10 including modular dose chambers 40a, 40b and attached transfer modules 50a, 50b for coupling with a drug chamber 30. The medication delivery system 10 may also comprise interchangeable dose chambers 40a, 40b and attached transfer modules 50a, 50b for coupling with the drug chamber 30. The dose chambers 40a, 40b and attached transfer chambers 50a, 50b may each comprise a fitting 76a, 76b configured to couple to a corresponding fitting 75 disposed on the drug chamber as described above with respect to FIG. 27. The dose chambers 40a, 40b may comprise the same or different interior volumes 41a, 41b. The transfer modules 50a, 50b may be configured to transfer the same or different specific or specific ranges of transfer volumes. Users may be provided one or more suitable dose chambers 40a, 40b and attached transfer modules 50a, 50b for the transfer and/or dosage volume needed. In some embodiments, a method of providing a medication treatment comprises providing a dose chamber 40a, 40b and attached transfer module 50a, 50b configured to provide an appropriate transfer volume for a prescribed dosage. In this or another embodiment, a method of providing a medication treatment may comprise providing a drug chamber 30 as described herein containing multiple dosages of a prescribed medication treatment. The drug chamber 30 may be sealed as to not spill or become contaminated during shipping or delivery.

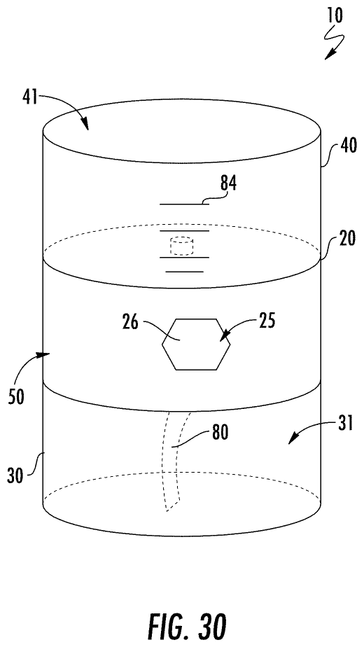

FIGS. 30-34 illustrate various embodiments of the medication delivery system 10 wherein the medication transfer device 20 comprises a modular container configuration. With reference to FIG. 30, the medication transfer device 20 includes a drug chamber 30, dose chamber 40, and transfer module 50 for providing transfer of medication through a transfer port (not visible) that extends through transfer module 50 from the interior volume 31 of the drug chamber 30 to the interior volume 41 of the dose chamber 40. In the illustrated embodiment, a hose 70 extends through the interior volume 31 of the drug chamber to interface with the transfer module 50. In some embodiments, an upper hose 70a may optionally extend into the interior volume 41 of the dose chamber 40. The chambers 30, 40 may comprise generally liquid impermeable material such as glass; plastic such as PVC, vinyl, polypropylenes (PP), polyethylenes (PE), polyethylene terephthalates (PET) such as boPET, or other plastics; ceramic; metal; or combinations thereof.

The medication transfer device 20 is configured to contain multiple dosages of medication within the drug chamber 30 and is operable to transfer a transfer volume of the medication into the interior volume 41 of the dose chamber 40. The transfer volume may be a predetermined, selectable, or customizable volume of medication according to a configuration of the transfer module 50 or device configuration, as described in more detail elsewhere herein. In various embodiments, the transfer module 50 may include a transfer valve (not shown) that may be selectively opened and closed, e.g., via user interaction with a user interface 25 or by assembling the device 20. In a further example, the transfer module 50 includes a gate valve. Control of the transfer valve may be used to prevent unwanted initiation of the transfer mechanism leading to unwanted transfer of medication and tampering by children. The medication transfer device 20 may be configured to utilize any suitable transfer mechanism, such as those described herein. The transfer module 50 may comprise a manual pump or a pump that couples to the output of a motor configured for assisted or automated pumping. In various embodiments, the transfer module 50 includes a valve configuration, bladder, and/or transfer chamber as described above with respect to FIGS. 19A-21C.

The transfer module 50 may include a shutoff valve or stop check-valve. In some embodiments, users may interface with operations of the valve at a user interface 25. The user interface 25 may comprise one or more switches, push buttons, knobs, or soft buttons, for example. The user interface 25 may include or operatively couple with, e.g., be operable to control, an actuator for opening and/or closing the valve. In one embodiment, the user interface 25 includes a control component that may be pushed, pulled, rotated, slid, switched, or otherwise actuated to cause opening or closing of the transfer port 51 (see FIG. 22). Actuation may cause a physical barrier to obstruct or reveal the transfer port 51. In some embodiments, initiation of transfer operations at the user interface 25 may open the valve for transfer operations. For example, initiation of transfer operations may not require additional steps that cause opening and closing of the valve. In one example, a user interface 25 may include button that initiates a pump to transfer medication. The pump may be electrically powered and/or manually powder, e.g., depressing the button may compress a bladder or bulb for hand pumping. However, in some embodiments, the transfer module 50 may maintain closure of the valve until a key, code, button sequence, or child-resistant feature has been overcome. As described in more detail below, in some embodiments, child-resistant features may include installation of the dose chamber 40, transfer module 50, or both. The user interface 25 may be distributed to locations other than the transfer module 50.

With continued reference to FIG. 30, as introduced above, the user interface 25 may include a button or other control component, which is generally referred to as an actuator 26 herein, wherein actuation of the actuator 26 initiates or drives transfer of medication from the drug chamber 30 to the dose chamber 40 through the transfer port. The actuator 26 may directly control operations by physically translating actuation to move a pump, move a transfer chamber, or translate valves, for example. In some embodiments, the actuator 26 may provide control instructions via an electrical circuit to control power to components such as an electric pump or solenoid. In further embodiments, the actuator 26 may provide control instructions to a control system processor that processes the instruction and executes a corresponding action based on programmed software. In one embodiment, actuation may cause a measurement component of the transfer module 50 to pump in or receive medication from the interior volume 31 of the drug chamber 30 and/or to pump out or release medication into the interior volume 41 of the dose chamber 40, which may comprise a measured portion of medication. Actuator 26 may be actuated to initiate a powering of a pump during actuation or for a predetermined time following actuation, e.g., based on a transfer volume entered by the user at the user interface 25. In one example, the actuator 26 or another user interface control component may be manipulated to specify a desired transfer volume that is used to determine pumping time. In one embodiment, actuator 26 may comprise a manual pump whereby actuation drives pumping of medication.

As introduced above, a mechanism for selectively opening and closing the transfer port 51, or allowing or preventing transfer, may be operated manually, electronically, and/or via motor assistance, which may be automated, e.g., operation of an switch, button, or actuator corresponding to initiation of medication transfer, or otherwise selectable operation by a user, e.g., operation of a switch, button, or actuator corresponding to powering a motor configured to open or close a valve or manipulate a barrier or structure to open or close the transfer port.

In various embodiments, the transfer module 50 comprises a control system including a processor configured to execute pumping operations to provide precise transfer and/or dosage volumes. For example, the control system may include a user interface 25 comprising a touch screen, one or more hard or soft buttons, knobs, switches, or other control components, which may comprise actuators 26 as noted above. The control system may include computer readable memory storing instructions that when executed by the processor execute operations of the control system. The control system may be operable to control operations of a motor and pump for pumping medication into the dose chamber 40. In one embodiment, the control system includes one or more sensors such as depth sensors, flow rate sensors, pressure sensors, weight sensors, optical sensors, or other sensors configured for determining a transfer volume. A user may enter a desired transfer or dosage volume into the user interface. The control system may then control operations of the pump to provide the entered volume.

FIG. 30 also illustrates an example of a medication transfer device 20 including a dose chamber 40 having measurement markings 42. A user may transfer medication to the dose chamber 40 and use the measurement markings 42 to measure a desired amount of transferred medication.

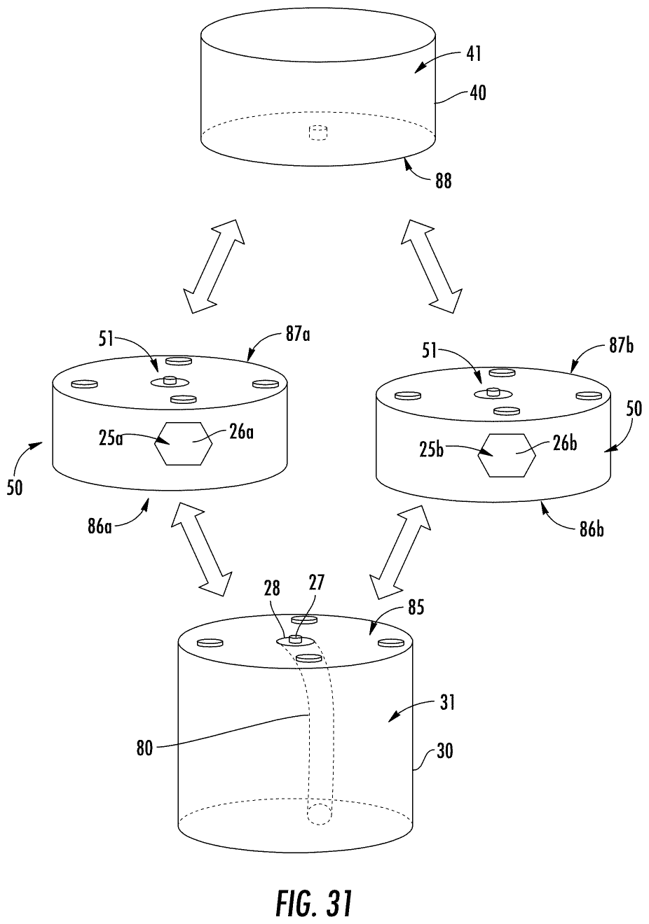

As introduced above, one or more components of the medication delivery system 10 may comprise modular and/or interchangeable components. With reference to FIG. 31, a medication delivery system 10 may comprise modular assembly of a drug chamber 30, dose chamber 40, and transfer module 50a, 50b. As shown, multiple interchangeable transfer modules 50a, 50b may be provided. In some embodiments, transfer modules 50a, 50b may be configured to transfer specific or specific ranges of transfer volumes.

Users may be provided one or more suitable transfer modules 50a, 50b for the transfer volume needed. In some embodiments, a method of providing a medication treatment comprises providing a transfer module 50a, 50b configured to provide an appropriate transfer volume for a prescribed dosage. In this or another embodiment, a method of providing a medication treatment may comprise providing a drug chamber 30 as described herein containing multiple dosages of a prescribed medication treatment. The drug chamber 30 may be sealed as to not spill or become contaminated during shipping or delivery. The drug chamber 30 may include a fitting 85 configured to couple to a corresponding fitting 86a, 86b of a transfer module 50a, 50b, which may include fittings disposed on a plurality of interchangeable transfer modules 50a, 50b. A drug port 28 may be positioned near fitting 85, around the area of coupling, for alignment and coupling with the transfer port 51. In some embodiments, the hose 80 couples to a closure valve 27 at or around the drug port 28. The drug port 28 and transfer port 51 may align when coupled. The fitting 86a, 86b of the transfer module 50a, 50b may engage or associate with the closure valve 27, hose 80, or extension thereof. In some embodiments, the engagement or association may result in providing control of the operation of closure valve 27 to the transfer module 50a, 50b, e.g., via an operation provided at user interface 25 utilizing an interface component such as actuator 26a, 26b. In the illustrated embodiment, coupling the fitting 85 to fitting 86a, 86b does not open the closure valve 27; however, in other embodiments, coupling the fittings 85, 86a, 86b opens the closure valve 27. In one embodiment, compressing the drug chamber 30 fitting 85 and the transfer module 50 fitting 86a together or relatively rotating the fittings 85, 86a may initiate opening or the ability to open the closure valve 27, e.g., via user interface. In one such example, the transfer module 50a, 50b seals or blocks the transfer port utilizing a valve, e.g., as described herein, to prevent undesirable transfer of medication until initiation of transfer. Dose chamber 40 may include a fitting 88 configured to couple to a complementary fitting 87a, 87b of the transfer module 50a, 50b, which may include fittings disposed on a plurality of interchangeable transfer modules 50a, 50b. Coupling the dose chamber 40 and transfer module 50a, 50b may align a port defined in the fitting 88 of the dose chamber 40 with the transfer port extending through the transfer module 50a, 50b. In various embodiments, the transfer module 50a, 50b is prevented for opening the transfer port or closure valve 27 unless properly coupled to the drug chamber 30, dose chamber 40, or both. Thus, in one example, operation of a transfer actuator or otherwise initiating transfer operations at the user interface 25 may be prevented if the transfer module 50a, 50b is coupled to the drug chamber 30 but not properly coupled to the dose chamber 40.

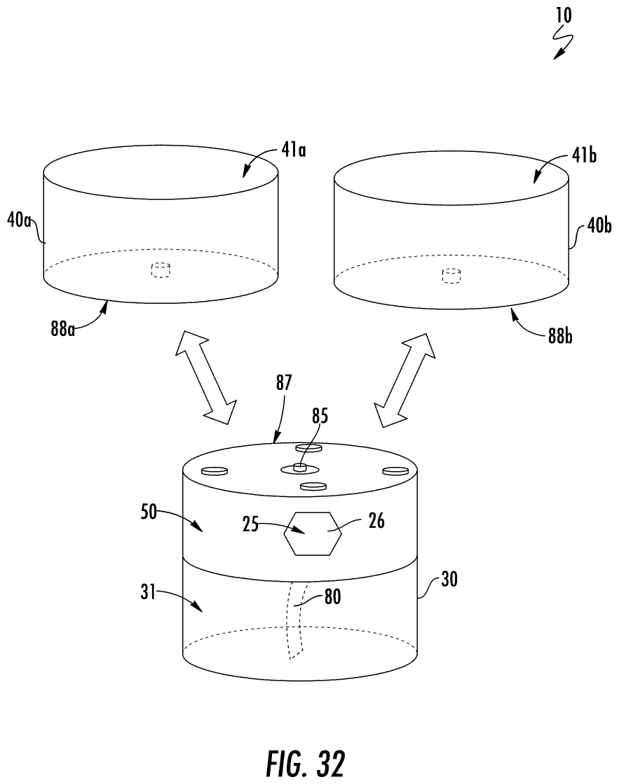

FIG. 32 illustrates a further example of a modular medication delivery system 10 including modular dose chambers 40a, 40b. The medication delivery system 10 may also comprise interchangeable dose chambers 40a, 40b. The dose chambers 40a, 40b may comprise the same or different interior volumes 41a, 41b. The dose chambers 40a, 40b may each comprise a fitting 88a, 88b configured to couple to a corresponding fitting 87 disposed on the transfer module 50 as described above with respect to FIG. 31. The drug chamber 30 may be sealed as to not spill or become contaminated during shipping or delivery as described above with respect to FIG. 31. Transfer operations may similarly be prevented until the transfer module 50 is properly coupled to the dose chamber 40. In various embodiments, users may be provided drug chambers containing medication and a transfer module 50 for transferring a transfer dose corresponding to a prescribed dosage. The user may couple the dose chamber 40 and transfer module 50 to transfer and thereby dispense the medication from the drug chamber 30.

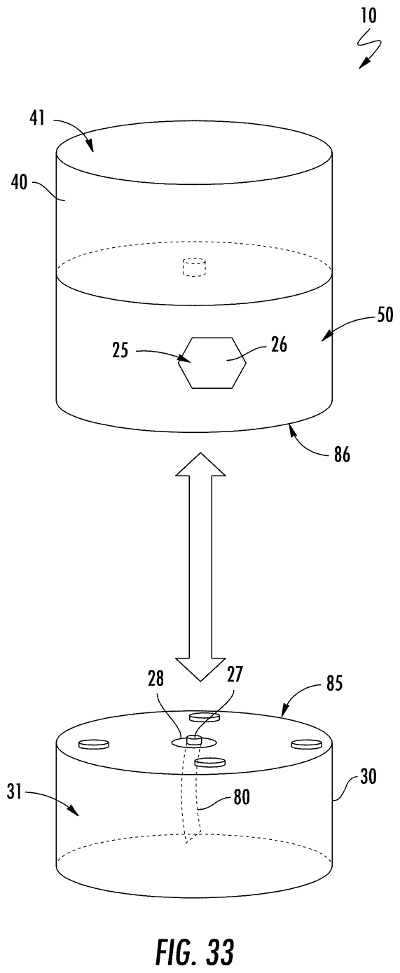

FIG. 33 illustrates a further example of a modular medication delivery system 10 including modular dose chambers 40a, 40b and attached transfer modules 50a, 50b for coupling with a drug chamber 30. The medication delivery system 10 may also comprise interchangeable dose chambers 40a, 40b and attached transfer modules 50a, 50b for coupling with the drug chamber 30. The dose chambers 40a, 40b and attached transfer chambers 50a, 50b may each comprise a fitting 86a, 86b configured to couple to a corresponding fitting 85 disposed on the drug chamber as described above with respect to FIG. 31. The dose chambers 40a, 40b may comprise the same or different interior volumes 41a, 41b. The transfer modules 50a, 50b may be configured to transfer the same or different specific or specific ranges of transfer volumes. Users may be provided one or more suitable dose chambers 40a, 40b and attached transfer modules 50a, 50b for the transfer and/or dosage volume needed. In some embodiments, a method of providing a medication treatment comprises providing a dose chamber 40a, 40b and attached transfer module 50a, 50b configured to provide an appropriate transfer volume for a prescribed dosage. In this or another embodiment, a method of providing a medication treatment may comprise providing a drug chamber 30 as described herein containing multiple dosages of a prescribed medication treatment. The drug chamber 30 may be sealed as to not spill or become contaminated during shipping or delivery as described above with respect to FIG. 31.

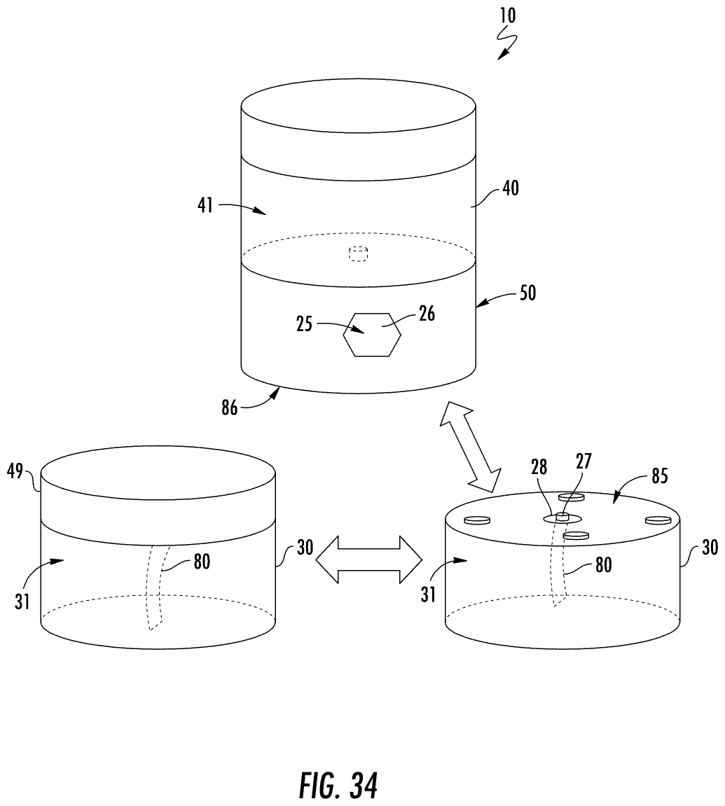

The embodiment illustrated in FIG. 34 is similar to that of described with respect to FIG. 31 and illustrates an example of a medication transfer device 20 having a lid 49 for covering the dose chamber 40. The lid 49 may include a fitting for coupling with a complementary fitting disposed on the dose chamber 40. In various embodiments, the lid 49 coupling with the dose chamber 40 includes a child-resistant features configured to resist decoupling by children, e.g., in compliance with the Poison Prevention Packaging Act or as otherwise with the Federal Drug Administration or U.S. Consumer Product Safety Commission. For example, the decoupling may require local squeeze force applied while turning, holding fitment while turning, pushing down while turning, pushing down into limited fitment gap followed by turning to secondary fitment gap, aligning cap and ring followed by pushing cap toward ring and turning, localized pushing up to remove, localized pressing down then pulling up at arrow, turning cap to stop and then lifting and continuing opening, localized pushing in while turning where force must be applied to designated place on closure, or other child-resistance mechanisms, which may include others described herein. Additional child-resistant features may include lug finish closures or continuous threads. FIG. 34 also illustrates a drug chamber 30 fitted with a lid 32. The lid 32 may provide anti-tampering capabilities during shipping or delivery and may be in addition to or as an alternative to a closure valve. The lid 32 may include a fitting configured to couple with a complimentary fitting disposed on the drug chamber 30. In one example, the lid 32 comprises a cap or over-cap that couples to the drug chamber 30 via a child-resistant mechanism, such as any identified herein. The dose chamber 40 may include a lid 49.

The medication delivery system may be utilized through the mail wherein a component such as a drug chamber containing medication is sent by a pharmacy, health provider, drug store or retailer to a caregiver or patient. The drug chamber may be protected during shipping from tampering herein and may further comprise child-resistant packaging as described herein. Access to or transfer of medication contained in the drug chamber may only be reasonably accessed by utilization of the system. Users may be sent transfer modules together with or separately of drug chambers. The transfer modules may be configured for use to transfer a prescribed dosage. In some embodiments, users may utilize any of a number of interchangeable transfer modules that may be configured to transfer different volumes of medication. In one embodiment, the medication delivery system includes a refill container. A refill container may include a bag, bottle, jar, pouch, or other container for containing medication. Refill containers may be mailed or otherwise provided to users for charging or refilling a drug chamber. In some embodiments, the refill container may be configured to transfer all or a portion of its medication contents into a drug chamber. The refill container may be opened to release medication for delivery into the drug chamber, e.g., through a fill port. In some embodiments, a refill container may be configured to couple to a transfer mechanism component. For example, a hose or fitting may be coupled between the drug chamber and the refill container. A electrically powered or hand pump may be used to assist in transfer. In another example, a transfer mechanism may include the transfer mechanism of the medication transfer device. That is, is one example, the refill container may comprise a modular drug chamber for coupling to the transfer mechanism to selectively transfer medication into the dose chamber as described herein.

In various embodiments, the medication may be transferred without utilization of outside measuring devices such as spoons or measuring cups. In one embodiment, a method of dispensing medication to a patient includes dispensing the medication in a liquid contained in the drug chamber. The method may also include dispensing the dose chamber and/or the transfer module providing the capacity to transfer the liquid medication. The does device may comprise a child resistant container such as a child-resistant bag, child-resistant bottle, or other device that meets or exceeds the industry accepted definition of child-resistant or child-proof.

In various embodiments, the dose chamber may be configured for administration and include an extrusion nozzle, applicator, spout, or spray nozzle.

The medication transfer device may be dispensed for use with various medications. For example, the medications may comprise liquid, cream, lotion, or gel formats. Medications may be include or be dispensed for formulation of compounded compositions for topical administration.

In various embodiments, the medication comprises or is dispensed for combination and topical administration to an external surface of a mammal, such as a human. In some embodiments, the topical composition may be formulated to treat infections or suspected infections of tissues and may be topically administered to surface tissues comprising or adjacent tissues thereof, which may include nails, wounded tissue, mucosal surfaces of the vagina or anus, skin such as on hands, feet, scalp, torso, arms, legs, or other surface. Embodiments of the composition may also be formulated to be applied to nails, a vaginal orifice, or anal orifice. In various embodiments, such a composition or component thereof may be supplied in the drug chamber and may be referred to herein as a topical composition.

The topical composition may generally include an antimicrobial agent, e.g., antibacterial, antifungal, or antiviral, comprising one or more pharmaceuticals drugs. However, the topical composition or medication may alternately or additional include other or different classes of pharmaceuticals drugs such as corticosteroids. Some embodiments may include combinations of active agents described herein without the antimicrobial agent. The topical composition may include a carrier comprising one or more carrier components. Unless stated otherwise, carrier is intended to include carrier component such that use of the term carrier may refer to a component of the carrier and is not restrictive in that other carrier components may be included and the carrier component referred to as the carrier need not form a complete carrier. Indeed, a carrier may include a thickening agent added to a commercially available medicated carrier solution, lotion, or cream, alone or together with other carriers, to formulate a carrier with respect to the topical composition. Carrier may also be used interchangeably with the term base. The carrier may be liquid, semi-liquid, or solid. For example, the carrier may include an aqueous, organic, or inorganic solution, which may include a dispersion or suspension, cream, gel, ointment, lotion, emulsion, powder, or paste. The topical composition may be formulated to treat microbial infections, such as infections of the skin, nails, mucosal surfaces, and potentially internalized infections, e.g., via transdermal administration of antimicrobial agents.

Embodiments of the topical composition may include an antimicrobial agent selected from an antibacterial component, antifungal component, or both. In one embodiment, the antibacterial component may include an antiviral agent. As introduced above, the topical composition may comprise the antimicrobial agent alone or in combination with one or more additional active agents selected from antibacterial component, antifungal component, an anti-inflammatory agent, a steroid, an anti-allergic agent, an antimicrobial agent, an anti-depressant agent, a stimulant agent, a disinfectant agent, an anticonvulsant agent, a local anesthetic agent, or combinations thereof. In one embodiment, the topical composition includes additional active agents selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antimicrobial agent including an antifungal component, antibacterial component, or both alone or in combination with a steroid agent, antiviral agent, NSAID agent, antidepressant agent, anticonvulsant agent, analgesic agent, opiate or opioid agonist agent, keratolytic agent, or combination thereof.

It is to be appreciated that recitations herein of a particular active pharmaceuticals include pharmaceutically acceptable salts thereof whether or not specifically recited as such. Similarly, recitation of a particular active pharmaceutical salt may also include other pharmaceutically acceptable salts thereof whether or not specifically recited as such.

In various embodiments, the antimicrobial agent comprises an antifungal component, alone or in combination with an antibacterial components, wherein the an antifungal component includes one or more antifungal pharmaceutical drugs selected from one or more categories of antifungal components including azoles (imidazoles), antimetabolites, allylamines, morpholine, glucan synthesis inhibitors (echinocandins), polyenes, benoxaaborale; other antifungal/onychomycosis agents, and new classes of antifungal/onychomycosis agents. For example, the antifungal component may comprise one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. In some embodiments, the antibacterial component is selected from one or more azoles. In one example, the antifungal component is selected from itraconazole, voriconazole, or combination thereof. In various embodiments, the antimicrobial agent comprises an antifungal component selected from one or more antifungals comprising fluconazole, itraconazole, voriconazole, amphotericin, nystatin, clotrimazole, econazole, or ketoconazole.

In various embodiments, the topical composition may comprise between approximately 0.01% and approximately 20% by weight antifungal component, such as between approximately 0.01% and approximately 5%, approximately 0.01% and approximately 3%, approximately 0.01% and approximately 1%, approximately 0.01% and approximately 0.25%, approximately 0.01% and approximately 0.15%, approximately 0.05% and approximately 0.15%, between 0.1% and 10%, approximately 0.1% and approximately 0.5%, approximately 0.1% and approximately 0.2%, approximately 0.2% and approximately 0.8%, approximately 0.2% and approximately 0.6%, approximately 0.2% and approximately 0.4%, approximately 0.3% and approximately 1%, approximately 0.3% and approximately 0.8%, approximately 0.3% and approximately 0.6%, approximately 0.4% and approximately 1%, approximately 0.5% and approximately 1%, approximately 0.5% and approximately 8%, approximately 0.6% and approximately 1%, approximately 0.6% and approximately 0.8%, approximately 0.8% and approximately 1%, approximately 1% and approximately 3%, approximately 1% and approximately 10%, approximately 1% and approximately 8%, approximately 1% and approximately 5%, approximately 1% and approximately 3%, approximately 3% and approximately 10%, approximately 3% and approximately 8%, approximately 3% and approximately 5%, between 5% and 10%, approximately 5% and approximately 8%, approximately 6% and approximately 10%, approximately 6% and approximately 8%, approximately 7% and approximately 10%, approximately 8% and approximately 10%, approximately 10% and approximately 20%, approximately 10% and approximately 15%, approximately 10% and approximately 12%, approximately 12% and approximately 15%, or between approximately 15% and approximately 20% antifungal component by weight. In some embodiments, the amount of antifungal component by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical composition.

It is to be appreciated that the amount of medication initially supplied in the drug chamber is preferably greater than that transferred to the dose chamber in a transfer or dose volume.

In various embodiments, the topical composition comprises an antimicrobial agent including an antifungal component alone or in combination with an anti-inflammatory agent, an non-steroidal anti-inflammatory (NSAID) agent, an anti-allergic agent, an antimicrobial agent, an anti-depressant agent, a stimulant agent, a disinfectant agent, an anticonvulsant agent, a local anesthetic agent, or combination thereof, which may include one or more active pharmaceutical drugs of selected components or agents. In one embodiment, the topical composition includes one or more additional active agents selected from one or more anticonvulsants, nerve depressants, muscle relaxants, NMDA (N-Methyl-D-aspartate) receptor antagonists, opiate or opioid agonists, antidepressants, and/or other actives. In some embodiments, the topical composition comprises an antifungal component alone or in combination with an antibacterial component, antiviral component, steroid agent, NSAID agent, antidepressant agent, anticonvulsant agent, analgesic agent, opioid agent, keratolytic agent, or combination thereof, which may include one or more active pharmaceutical drugs of selected components or agents. In an above or another embodiment, the antimicrobial agent may further comprise an antibacterial component comprising one or more antibacterial pharmaceutical drugs, such as those identified herein.

The antimicrobial agent may comprise an antibacterial component alone or in combination with an antifungal component. In some embodiments, the antibacterial component comprises one or more enicillins, cephalosporins, fluoroquinolones, aminoglycosides, monobactams, carbapenems, macrolides, other antibacterial, or combination thereof. For example, the antibacterial component may include one or more antibacterial pharmaceutical drugs selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, methicillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilamide, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In some embodiments, the antibacterial component is selected from mupirocin, gentamycin, tobramycin, or combinations thereof. In one embodiment, the antibacterial component includes an aminoglycoside.

In various embodiments, the one or more antimicrobial agents comprises an antibacterial component selected from one or more antibacterials comprising vancomycin, ciprofloxacin, levofloxacin, azithromycin, clindamycin, doxycycline, mupirocin, ceftriaxone, colistimethate, tobramycin, cefepime, gentamicin, streptomycin, sulfamethoxazole/trimethoprim. In one example, the topical composition comprises linezolid, levofloxacin, ciprofloxacin, or combination thereof.

In various embodiments, the topical composition may comprise between approximately 0.01% and approximately 20% by weight antibacterial component, such as between approximately 0.01% and approximately 5%, approximately 0.01% and approximately 3%, approximately 0.01% and approximately 1%, approximately 0.01% and approximately 0.25%, approximately 0.01% and approximately 0.15%, approximately 0.05% and approximately 0.15%, between 0.1% and 10%, approximately 0.1% and approximately 0.5%, approximately 0.1% and approximately 0.2% approximately 0.2% and approximately 0.8%, approximately 0.2% and approximately 0.6%, approximately 0.2% and approximately 0.4%, approximately 0.3% and approximately 1%, approximately 0.3% and approximately 0.8%, approximately 0.3% and approximately 0.6%, approximately 0.4% and approximately 1%, approximately 0.5% and approximately 1%, approximately 0.5% and approximately 8%, approximately 0.6% and approximately 1%, approximately 0.6% and approximately 0.8%, approximately 0.8% and approximately 1%, approximately 1% and approximately 3%, approximately 1% and approximately 10%, approximately 1% and approximately 8%, approximately 1% and approximately 5%, approximately 1% and approximately 3%, approximately 3% and approximately 10%, approximately 3% and approximately 8%, approximately 3% and approximately 5%, between 5% and 10%, approximately 5% and approximately 8%, approximately 6% and approximately 10%, approximately 6% and approximately 8%, approximately 7% and approximately 10%, approximately 8% and approximately 10%, approximately 10% and approximately 20%, approximately 10% and approximately 15%, approximately 10% and approximately 12%, approximately 12% and approximately 15%, or between approximately 15% and approximately 20% antibacterial component by weight. In some embodiments, the amount of antibacterial component by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical composition.

In some examples, the topical composition comprises from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, from approximately 3.0% w/w to approximately 7.0% w/w, or from approximately 4.0% w/w to approximately 7.0% w/w of an antifungal component identified herein. For example, the topical composition may comprise itraconazole, voriconazole, fluconazole, or combination thereof. In an example, the topical composition comprises from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, from approximately 3.0% w/w to approximately 7.0% w/w, or from approximately 4.0% w/w to approximately 7.0% w/w of a first antifungal component identified herein and from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, approximately 3.0% w/w to approximately 7.0% w/w, or from approximately 4.0% w/w to approximately 7.0% w/w of a second antifungal component identified herein.

In one example, the topical composition comprises from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, approximately 3.0% w/w to approximately 7.0% w/w, or from approximately 4.0% w/w to approximately 7.0% w/w of an antibacterial component identified herein. In another example, the transfer volume or dosage volume of the topical composition comprises from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, approximately 3.0% w/w to approximately 7.0% w/w, or from approximately 4.0% w/w to approximately 7.0% w/w of a first antibacterial component identified herein and from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, approximately 3.0% w/w to approximately 7.0% w/w, or from approximately 4.0% w/w to approximately 7.0% w/w of a second antibacterial component identified herein. For example, the topical composition may comprise mupirocin and tobramycin, mupirocin and doxycycline, mupirocin and doxycycline hyclate, mupirocin and azithromycin, or mupirocin, doxycycline, and ketoconazole.

In one example, the topical composition comprises from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, approximately 3.0% w/w to approximately 7.0% w/w, or from approximately 4.0% w/w to approximately 7.0% w/w of an antifungal component identified herein and from approximately 0.3% w/w to approximately 3% w/w, approximately 0.5% w/w to approximately 2.5% w/w, approximately 1.0% w/w to approximately 9.0% w/w, approximately 2.0% w/w to approximately 8.0% w/w, approximately 3.0% w/w to approximately 7.0% w/w, or from approximately 4.0% w/w to approximately 7.0% w/w of an antibacterial component identified herein. For example, the antibacterial component may comprise doxycycline, tobramycin, ciprofloxacin, mupirocin, or combination thereof and the antifungal component may comprise ketoconazole, itraconazole, voriconazole, or combination thereof.

In some embodiments, the topical composition may comprise one or more excipients or additives. In an aspect, excipients or additives include, but are not limited to, the following: solvents, surfactants, humectants, preservatives, flavorings, stabilizers (including antioxidants), binders, and colorants.

In various embodiments, the topical composition comprises the antibacterial component alone or in combination with one or more additional active agents selected from an antifungal component, an antiviral agent, an anti-inflammatory agent, an non-steroidal anti-inflammatory (NSAID) agent, an anti-allergic agent, an antimicrobial agent, an anti-depressant agent, a stimulant agent, a disinfectant agent, an anticonvulsant agent, a local anesthetic agent, or combinations thereof, which may include one or more active pharmaceutical drugs of selected components or agents. In one embodiment, the topical composition includes additional active agents selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antibacterial component alone or in combination with an antifungal component, steroid agent, antiviral component, NSAID agent, antidepressant agent, anticonvulsant agent, analgesic agent, opioid agent, keratolytic agent, or combination thereof, which may include one or more active pharmaceutical drugs of selected components or agents. In various embodiments, the topical composition may comprise the antibacterial component alone or in combination with one or more antifungal components.

As introduced above, the topical composition may comprise one or more additional active agents. It will be appreciated that topical compositions herein may include or specifically exclude additional active agents. It will also be appreciated that topical compositions may exclude an antimicrobial agent and rather include one or more of the additional active agents described herein.

In various embodiments, the topical composition comprises the antimicrobial agent and a nonsteroidal anti-inflammatory drug (NSAID) agent. The NSAID agent may include one or more NSAIDS selected from oxicams, such as meloxicam or piroxicam; salicylic acid derivatives, such as aspirin, diflunisal, salsalate, or trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, or tolmetin; fenamates, such as meclofenamate; and/or COX-2 inhibitors, such as celecoxib, rofecoxib, or valdecoxib. In various embodiments, the topical composition may comprise between approximately 0.01% and approximately 20% by weight NSAID agent.

In some embodiments, the topical composition comprises the antimicrobial agent and a local anesthetic agent. The local anesthetic agent may be selected from lidocaine, prilocaine, benzocaine, or combination thereof. The local anesthetic agent may comprise between approximately 0.01% and approximately 15% by weight of the topical composition.

In an embodiment, the topical composition comprises the antimicrobial agent a steroid agent. In one example, the steroid agent comprises a corticosteroid selected from amcinonide, betamethasone dipropionate, betamethasone valerate, clobetasol propionate, desoximetasone, diflorasone diacetate, flurandrenolide, fluticasone propionate, fluocinonide, halcinonide, halobetasol propionate, mometasone furoate, triamcinolone acetonide, or combination thereof. In various embodiments, the topical composition comprises between approximately 0.001% and approximately 1% by weight steroid agent.

In various embodiments, the topical composition comprises the antimicrobial agent and a muscle relaxant agent comprising one or more muscle relaxants selected from baclofen, carisoprodol, chlorzoxazone, cyclobenzaprine, dantrolene, diazepam, metaxalone, methocarbamol, orphenadrine, quinine sulfate, tizanidine, and/or other muscle relaxants. In various embodiments, the topical composition comprises between approximately 0.001% and approximately 5% by weight muscle relaxant agent.

In some embodiments, the topical composition comprises the antimicrobial agent and an anticonvulsant or nerve depressant agent. The anticonvulsant or nerve depressant agent may comprise one or more nerve depressants and/or anticonvulsants selected from gabapentin, topiramate, lamotrigine, or combinations thereof. In various embodiments, the anticonvulsant or nerve depressant agent may comprise between approximately 0.01% and approximately 20% by weight of the topical composition.

In one embodiment, the topical composition comprises the antimicrobial agent and a NMDA receptor antagonist agent such as ketamine. In some embodiments, the topical composition may comprise an opiate or opioid agonist agent selected from tramadol; one or more C2 opiate agonists selected from oxycodone, morphine, methadone, hydromorphone, and fentanyl; one or more C3 opiate agonists selected from hydrocodone, codeine, propoxyphene, butalbital, and pentazocine; or any combination thereof.

In an embodiment, the topical composition comprises the antimicrobial agents a keratolytic agent selected form urea, salicylic acid, papain, or combinations thereof. For example, the topical composition may comprise the antimicrobial agent and urea. In various embodiments, the topical composition may comprise between approximately 1% and approximately 30% by weight urea.

The topical composition may be provided in a topical format, which may include a carrier for topical administration. In various embodiments, the topical composition may include a colloid or emulsion (o/w, w/o), cream, lotion, ointment, foam, aqueous or non-aqueous gel, aqueous or non-aqueous solution, which may include a dispersion, powder, nail lacquer, bath, or paste.

The topical composition may be administered topically by contacting an external surface of the body, which may include skin, e.g., intact, wounded, broken skin; nails; mucosal tissue lining a vaginal or anal orifice. The topical composition may be administered in a spray, coating, soak, powder, spread, or the like, for example, suitable to the topical format.

In some embodiments, the topical composition comprises a nail lacquer for direct application to nail tissue. A nail lacquer format may include one or more antimicrobial actives formulated for topical application to nail tissue. In some embodiments, a nail lacquer format may include additives such as thickening agents, plasticizers, polymers, volatile organic compounds, or other additives to promote effective localization of the medication following application. In some embodiments, a nail lacquer format may comprise a solution, which may be a suspension or mixture. In some embodiments, a nail lacquer format may lack traditional lacquer additives. In various embodiments, a nail lacquer format may comprise an aqueous solution formulated for application to a nail surface whereon the carrier evaporates or is absorbed. In some embodiments, a nail lacquer solution may have a fluid or semi-fluid consistency. In some embodiments, a carrier for a nail lacquer format may be thickened with a viscosity agent to increase viscosity for administration. In some embodiments, a nail lacquer format may comprise a solution comprising a cream, lotion, gel, or ointment.

Further to the above, in some embodiments, the topical composition comprises a treatment solution for a footbath, irrigation, or spray administration.

In various embodiments, the topical composition comprises an antimicrobial agent comprising an antifungal component comprising at least two antifungal pharmaceutical drugs, an antibacterial component comprising at least two antibacterial pharmaceutical drugs, or an antifungal component comprising one or more antifungal pharmaceutical drugs and an antibacterial component comprising one or more antibacterial pharmaceutical drugs.

In one example, the topical composition comprises mupirocin and/or azithromycin and an antifungal component selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. The antifungal component may comprise itraconazole and/or fluconazole, for example. In one embodiment, the topical composition comprises mupirocin and itraconazole, mupirocin and nystatin, or azithromycin and fluconazole. In another example, the topical composition comprises mupirocin and/or azithromycin and an additional antibacterial pharmaceutical drug identified herein.

In another example, the topical composition comprises an antifungal component including itraconazole and a second, different, antimicrobial pharmaceutical drug. For example, the topical composition may comprise an antibacterial component comprising the second antimicrobial pharmaceutical drug that includes one or more antibacterial pharmaceutical drugs selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, metronidazole, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In another example, the topical composition comprises an antifungal component including itraconazole and a second, different, antimicrobial pharmaceutical drug, comprising an additional antifungal pharmaceutical drug selected from an azole. In one example, the azole includes clotrimazole, econazole, fluconazole, isoconazole, ketoconazole, voriconazole, or combination thereof. In another example, the additional antifungal pharmaceutical drug is selected from antimetabolites, allylamines, morpholine, glucan synthesis inhibitors (echinocandins), polyenes, benoxaaborale; other antifungal/onychomycosis agents, and new classes of antifungal/onychomycosis agents. In another example, the additional antifungal pharmaceutical drug is selected from abafungin, albaconazole, amorolfin, anidulafungin, bifonazole, butenafine, butoconazole, candicidin, caspofungin, ciclopirox, fenticonazole, filipin, flucytosine, griseofulvin, haloprogin, hamycin, isavuconazole, micafungin, miconazole, naftifine, natamycin, omoconazole, oxiconazole, polygodial, posaconazole, ravuconazole, rimocidin, sertaconazole, sulconazole, terbinafine, terconazole, tioconazole, undecylenic acid, or a combination thereof. In another example, the additional antifungal pharmaceutical drug is selected from amphotericin b, nystatin, tolnaftate, or combination thereof.

In some embodiments, medication transfer system may be utilized to treat infections associated with MRSA, a Candida, such as Candida albicans, Candida auris, Candida glabrata, Candida krusei, or Candida tropicalis may include topically applying the topical composition to target skin or mucosal surface. In some examples, the antimicrobial agent may comprise an antifungal component comprising an azole. In one example, the antifungal component comprises itraconazole. In a further example, the topical composition comprises itraconazole oral solution. In a further example, the topical composition comprises itraconazole oral solution and a carrier, such as a diluent or base for compounding. The topical composition may also include one or more additional antifungal active drugs, an antibacterial component, and/or one or more additional active agents.

In some embodiments, the medication comprises a commercially available

In one embodiment, the medication comprises a commercially available Itraconazole Oral Solution or Levofloxacin Oral Solution. For example, Itraconazole Oral Solution may contain 10 mg of itraconazole per mL, solubilized by hydroxypropyl-β-cyclodextrin (400 mg/mL) as a molecular inclusion complex and may have a target pH of 2. Accordingly, the solution may have a low pH of approximately 2. Other ingredients may include hydrochloric acid, propylene glycol, purified water, sodium hydroxide, sodium saccharin, sorbitol, cherry flavor, and caramel flavor. It will be appreciated that oral solutions comprising other flavorings may also be used if they become available. Similarly, other pH adjusting agents may also be used if they become available. Levofloxacin oral solution, which is available in the United States in 25 mg/mL strength formulations. Such oral solutions also include inactives such as vehicles, solvents, stabilizers, coloring agents, or flavoring agents. In one example, levofloxacin oral solution contains, in addition to levofloxacin, artificial and natural flavors, ascorbic acid, benzyl alcohol, caramel color, glycerin, hydrochloric acid, propylene glycol, purified water, sucralose and sucrose. As another example, levofloxacin oral solution contains the following inactive ingredients: artificial bubble gum flavor, artificial grape flavor, ascorbic acid, benzyl alcohol, glycerin, hydrochloric acid, PFC Bitter Mask F9885, propylene glycol, purified water, saccharin sodium, and sucrose. Sodium hydroxide may be used to adjust pH (between approximately 5.0 to approximately 6.0). Levofloxacin is also currently administered parenterally via intravenous injection. Levofloxacin for injection is commercially available in various strengths and volumes. For example, levofloxacin for injection is currently available in 500 mg/20 mL strength, 20 mL volume single use container, and in 250 mg/50 mL strength, 50 mL, 100 mL, and 150 mL single-use containers. Additional active agents may include one or more antifungal actives, antibacterial actives, or both. Such additional antifungal component may be present in a combined amount between approximately 0.01% and approximately 20% by weight, such as between approximately 0.01% and approximately 5%, approximately 0.01% and approximately 2% by weight, approximately 0.05% and approximately 2%, approximately 0.1% and approximately 2%, approximately 0.5% and approximately 2%, approximately 1% and approximately 2%, approximately 2% and approximately 7%, or approximately 0.05%, approximately 0.1%, approximately 0.5%, approximately 1%, approximately 2%, approximately 3%, approximately 4%, approximately 5%, less than approximately 5%, or greater than approximately 10% itraconazole by weight. The medication may include itraconazole, e.g., itraconazole oral solution, and an antibacterial component comprising the second antimicrobial pharmaceutical drug selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, metronidazole, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof.

Additionally or alternatively, additional actives may include other active agents such as one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between approximately 0.01% and approximately 25% by weight, such as between approximately 1% and approximately 10%. The topical composition including the medication comprising itraconazole may be utilized as part of a treatment of a microbial infection. In one example, the topical composition may be topically administered to infected skin forming the outer body covering of a subject or to mucosal tissue of the vagina or anus to treat a microbial infection. For example, the topical composition may comprise a solution or suspension for topical administration in a hand or footbath or by irrigation. In another example, the topical composition comprises a nail lacquer for administration to nails. In some embodiments, the topical composition may be utilized as a wound treatment and administered to broken or unbroken skin or mucosal tissue as indicated above and elsewhere herein.

In an example, the antimicrobial agent includes an antifungal component comprising fluconazole and the method of formulating the topical composition comprises combining a carrier and a commercially available fluconazole, such as Fluconazole in Dextrose Injection Solution; Fluconazole in Sodium Chloride Injection Solution, Ketoconazole Suspension, Clotrimazole Liquid, Voriconazole Oral Suspension, ciprofloxacin, such Ciprofloxacin Hydrochloride Solution/Drops; Ciprofloxacin Hydrochloride Suspension; Azithromycin for Oral Suspension, USP, which may be supplied for suspension in 100 mg/5 mL or 200 mg/5 mL; Clindamycin Phosphate Suspension; Clindamycin Phosphate Injection Solution; Cefepime Hydrochloride Injection Solution; Sulfamethoxazole and Trimethoprim Suspension; Ciprofloxacin Cream, Ciprofloxacin Ointment, Clindamycin Cream, Clindamycin Ointment, Clindamycin Gel, Gentamycin drops, Gentamycin Spray, Gentamycin Cream, Gentamycin Ointment, Levofloxacin Injection Solution, Levofloxacin Drops, Mupirocin Ointment, Mupirocin Cream, Tobramycin Ophthalmic Ointment, Tobramycin Ophthalmic Drops, and/or Tobramycin Otic Drops, linezolid oral suspension; 2% erythromycin solution.

In an aspect, transfer or dosage volume may include between approximately 1 mg and approximately 2000 mg, such as between approximately 100 mg and approximately 1000 mg, approximately 250 mg and approximately 750 mg, approximately 250 mg and approximately 500 mg, or approximately 750 mg levofloxacin in a dosage volume. According to various embodiments, a dosage volume may be approximately 20 mL to approximately 4 L, such as approximately 30 mL to approximately 2 L, approximately 40 mL to approximately 1.5 L, or approximately 40 mL to approximately 1 L. Dosage volumes greater than 4 L may also be used, e.g. greater than 5 L, greater than 10 L, greater than 15 L, or greater than 20 L. In an aspect, transfer or dosage volume may include between approximately 0.01 mg/mL and approximately 24 mg/mL levofloxacin, such as approximately 0.5 mg/mL and approximately 2 mg/mL, approximately 1 mg/mL and approximately 10 mg/mL, approximately 5 mg/mL and approximately 13 mg/mL, or approximately 10 mg/mL and approximately 20 mg/mL. In an aspect, the treatment solution may include greater than a 25 mg/mL levofloxacin concentration. In an aspect, tobramycin may be substituted for levofloxacin.

In an aspect, a transfer or dosage volume may include between approximately 10 mg and approximately 300 mg, such as between approximately 40 mg and approximately 250 mg, approximately 50 mg and approximately 200 mg, approximately 50 mg and approximately 125 mg, approximately 150 mg and approximately 200 mg itraconazole in a dosage volume. In an aspect, a transfer or dosage volume for a small treatment area may contain between approximately 1 mg and approximately 30 mg, such as between approximately 5 mg and approximately 25 mg, approximately 10 mg and approximately 25 mg, approximately 10 mg and approximately 25 mg, approximately 15 mg and approximately 20 mg itraconazole in a dosage volume.

In various embodiments, transfer or dosage volume may include itraconazole oral solution, 10 mg/mL, alone or with a diluent, wherein the itraconazole oral solution is in an amount between approximately 1 mL and approximately 25 mL in a dosage volume. For example, the amount of itraconazole oral solution in a dosage volume may be between approximately 1 mL and approximately 3 mL, approximately 3 mL and approximately 25 mL, approximately 5 mL and approximately 20 mL, approximately 5 mL and approximately 10 mL, approximately 10 mL and approximately 20 mL, approximately 15 mL and approximately 25 mL, or greater than approximately 1 mL, approximately 2 mL, approximately 5 mL, approximately 10 mL, approximately 15 mL, approximately 20 mL, or less than approximately 25 mL.

According to various embodiments, a dosage volume may be between approximately 20 mL and approximately 4 L, such as approximately 30 mL and approximately 2 L, approximately 40 mL and approximately 1.5 L, or approximately 40 mL and approximately 1 L. Dosage volumes greater than 4 L may also be used, e.g. greater than 5 L, greater than 10 L, greater than 15 L, or greater than 20 L. In some embodiments, a dosage volume for a small treatment area may comprise between approximately 1 mL and approximately 5 mL, such as approximately 1 mL and approximately 4 mL, approximately 1 mL and approximately 3 mL, approximately 2 mL and approximately 5 mL, approximately 2 mL and approximately 3 mL, or between approximately 3 mL and approximately 5 mL. A transfer volume may be added to a diluent to obtain a full dosage volume including diluent.

In an aspect, a transfer or dosage volume may include between approximately 0.01 mg/mL and approximately 9.5 mg/mL itraconazole, such as between approximately 0.5 mg/mL and approximately 9 mg/mL, approximately 1 mg/mL and approximately 8 mg/mL, approximately 2 mg/mL and approximately 7 mg/mL, or approximately 10 mg/mL and approximately 20 mg/mL. In an embodiment, the treatment solution may include greater than a 10 mg/mL itraconazole concentration. In one embodiment, itraconazole is replaced with tobramycin.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

Various elements described herein have been described as alternatives or alternative combinations, e.g., in a lists of selectable actives, ingredients, or compositions. It is to be appreciated that embodiments may include one, more, or all of any such elements. Thus, this description includes embodiments of all such elements independently and embodiments including such elements in all combinations.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “approximately” are intended to include +/−10% of the number modified.

What is claimed is:

1. A method of supplying a medication for use with a medication transfer system, the method comprising:
supplying a liquid medication into an interior volume of a drug chamber; and
dispensing the drug chamber to a patient or caregiver,
wherein the drug chamber includes a fitting for coupling to a corresponding fitting of a transfer module, wherein the drug chamber includes a drug port and the transfer module includes a transfer port configured to align with the drug port when the drug chamber and transfer module are coupled at their fittings, and wherein the transfer module is operable to open the drug port to allow transfer a portion of the liquid medication through the transfer port into a separate dose chamber.

2. The method of claim 1, wherein the liquid medication is a prescription medication comprising an anti-infective and/or corticosteroid.

3. The method of claim 1, wherein the liquid medication comprises levofloxacin or itraconazole.

4. The method of claim 1, wherein the liquid medication comprises levofloxacin 25 mg/mL oral solution.

5. The method of claim 1, wherein the liquid medication comprises itraconazole 10 mg/mL oral solution.

6. The method of claim 1, wherein the drug chamber includes a hose extending from within its interior volume to the drug port.

7. A method of preparing a measured medication dosage, the method comprising:
transferring a transfer volume of liquid medication from a drug chamber through a transfer port and into a dose chamber, wherein a transfer module is positioned to at least partially control transfer of the liquid medication through the transfer port, and wherein the transfer volume is less than the volume of liquid medication within the drug chamber when transfer begins.

8. The method of claim 7, wherein the dose chamber is configured for measurement of the transfer volume.

9. The method of claim 8, wherein dose chamber includes measurement markings corresponding to the transfer volume.

10. The method of claim 7, wherein the transfer module is configured for measurement of the volume of transferred medication.

11. The method of claim 11, wherein the transfer module includes a user interface comprising a control actuator, and wherein transferring comprises actuating the control actuator to initiate the transfer of the liquid medication.

12. The method of claim 7, wherein the drug chamber is child-resistant.

13. The method of claim 7, wherein the liquid medication is a prescription medication comprising an anti-infective and/or corticosteroid.

14. The method of claim 13, wherein the liquid medication comprises levofloxacin 25 mg/mL oral solution, itraconazole 10 mg/mL oral solution, or both.

15. A method of supplying a medication together with a medication transfer system, the method comprising:
dispensing a medication transfer device comprising:
a drug chamber having an interior volume,
a dose chamber having an interior volume, and
a transfer module including a transfer port between the interior volumes of the drug chamber and the dose chamber; and
dispensing a liquid medication within the interior volume of the drug chamber when dispensing the drug chamber,
wherein the liquid medication is selectively transferable from the interior volume of the drug chamber to the interior volume of the dose chamber, and
wherein the medication transfer device is configured for measurement of a volume of the transferred liquid medication.

16. The method of claim 15, where the medication transfer device comprises a bag that is partitioned to define the drug chamber and dose chamber.

17. The method of claim 15, wherein the medication transfer device comprises a child-resistant device.

18. The method of claim 15, wherein the medication transfer device comprises a child-resistant bottle or child-resistant bag.

19. The method of claim 15, wherein the liquid medication is a prescription medication comprising an anti-infective and/or corticosteroid.

20. The method of claim 15, wherein the liquid medication comprises levofloxacin 25 mg/mL oral solution, itraconazole 10 mg/mL oral solution, or both.
